(19) United States
(12) Patent Application Publication
(10) Pub. No.: US 2024/0037743 A1
(43) Pub. Date: Feb. 1, 2024

(54) SYSTEMS AND METHODS FOR EVALUATING EMBRYO VIABILITY USING ARTIFICIAL INTELLIGENCE

(71) Applicant: Alife Health Inc., Cambridge, MA (US)

(72) Inventors: Kevin LOEWKE, Menlo Park, CA (US); Mark LOWN, Castro Valley, CA (US); Melissa TERAN, San Francisco, CA (US); Paxton MAEDER-YORK, Cambridge, MA (US)

(21) Appl. No.: 18/453,968

(22) Filed: Aug. 22, 2023

Related U.S. Application Data
(63) Continuation of application No. PCT/US2022/018743, filed on Mar. 3, 2022.
(60) Provisional application No. 63/256,332, filed on Oct. 15, 2021.
(60) Provisional application No. 63/157,433, filed on Mar. 5, 2021.

(51) Int. Cl. G06T 7/00; G16H 10/60; G16H 50/20; G16H 50/30; G16H 80/00

(52) U.S. Cl. CPC G06T 7/0012; G16H 10/60; G16H 50/20; G16H 50/30; G16H 80/00; G06T 2207/30044; G06T 2207/20084; G06T 2207/10056

(57) ABSTRACT

Systems and methods for predicting viability of one or more embryos is described herein. In some variations, a method may include receiving a single image of the embryo via a real-time communication link with an image capturing device and generating a viability score for the embryo by classifying the single image via at least one convolutional neural network. In some variations, a method may include receiving a plurality of single images, where each single image depicts a different respective embryo of a plurality of embryos, generating a viability score for each embryo by classifying each single image via at least one convolutional neural network, and ranking the plurality of embryos based on the viability scores for the plurality of embryos.

### CROSS-REFERENCE TO RELATED APPLICATIONS

This application is a continuation of International Patent Application No. PCT/US2022/018743, filed Mar. 3, 2022, which claims priority to U.S. Provisional Patent Application No. 63/256,332, filed Oct. 15, 2021, and U.S. Provisional Patent Application No. 63/157,433, filed Mar. 5, 2021, each of which is hereby incorporated by reference in its entirety.

### TECHNICAL FIELD

This invention relates generally to the field of evaluating embryo viability.

### BACKGROUND

In vitro fertilization (IVF) is a widely known assisted reproductive technology. IVF involves several complex steps such as ovarian stimulation, oocyte retrieval, oocyte fertilization, embryo culture, embryo selection, and embryo transfer. Typically, embryos are cultured to the blastocyst stage (e.g., the embryo transfer stage). That is, following the oocyte retrieval and fertilization, embryos are cultured until there is a clear differentiation into the inner cell mass and trophectoderm structures. Less competent embryos often arrest their development prior to the blastocyst stage. Generally, a cohort of embryos may make it to the blastocyst stage. Therefore, embryos that survive to the blastocyst stage need to be assessed before an embryo is selected for transfer. Based on the assessment, a single embryo (or, in rare cases multiple embryos) may be selected for transfer.

Accordingly, embryo selection is an important aspect of the IVF process. Traditionally, embryo selection is performed by an embryologist manually inspecting and assessing embryos. The embryologist may assign grades to embryos by inspecting embryos under a microscope. The embryologist may assess features such as the degree of blastocyst expansion, the quality of the inner cell mass, and the quality of the trophectoderm in order to grade embryos. However, manually grading embryos can be a highly subjective process. Different embryologists may grade an embryo differently based on their respective manual inspections. Studies have found that manual inspection and grading may be often be an intuition driven approach. Therefore, the grades may vary drastically depending on the embryologist inspecting the embryos.

More recently, non-manual techniques have been explored in order to make the process of embryo selection more consistent. However, these existing techniques have failed to gain widespread adoption. For example, time-lapse imaging that captures a sequence of images of an embryo in a periodic manner has been extensively studied. However, time-lapse imaging requires specialized microscopes that tend to be expensive. The high cost of installation has inhibited clinics and laboratories from adopting the technology. Another technique that has been researched recently is preimplantation genetic testing for aneuploidy (PGT-A). Existing PGT-A tests are invasive tests. Concerns have been raised about embryo health following these tests. Additionally, existing PGT-A tests merely identify euploid and aneuploid embryos. While it is known that aneuploid embryos are unlikely to have a successful pregnancy outcome, it is not necessary for all euploid embryos to lead to a successful pregnancy outcome. Thus, existing PGT-A tests may still not entirely solve the problem of assessing embryo viability.

Therefore, there is an unmet need for new and improved methods to standardize the grading of embryos and improve the accuracy of predicting the viability of embryos. Furthermore, there is an unmet need for new and improved methods of evaluating embryo viability that are cost-effective, easy to implement, and easy to adopt.

### SUMMARY

Generally, in some variations, a computer-implemented method for predicting viability of an embryo may include receiving a single image of an embryo and generating a viability score for the embryo by classifying the single image via at least one convolutional neural network, where the viability score represents predicted viability of the embryo. In some variations, the single image that is classified via the at least one convolutional neural network is not part of a time series of images. The viability score may, for example, represent predicted likelihood of the embryo reaching clinical pregnancy (e.g., the likelihood of the embryo reaching clinical pregnancy may be associated with an outcome of a fetal cardiac activity), likelihood of the embryo reaching live birth, and/or the like. In some variations, the viability score may be at least in part on data associated with a patient, such as age, body mass index, day of image capture, and donor status. Once generated, the viability score may be stored in a database associated with a patient (e.g., patient in which the embryo may be implanted), and/or communicated to a patient, clinician, user of the image capturing device, etc.

For example, in some variations, a computer-implemented method may include receiving a single image over a real-time communication link with an image capturing device, cropping the single image to a boundary of the embryo via a first convolutional neural network, and generating a viability score for the embryo by classifying the single image via at least a second convolutional neural network. In some variations, the single image that is classified is not part of a time series of images. As described above, the viability score may, for example, represent predicted likelihood of the embryo reaching clinical pregnancy (e.g., the likelihood of the embryo reaching clinical pregnancy may be associated with an outcome of a fetal cardiac activity). As another example, the viability score may represent likelihood of the embryo reaching live birth. In some variations, the viability score may be at least in part on data associated with a patient, such as age, body mass index, day of image capture, and donor status. Once generated, the viability score may be stored in a database associated with a patient (e.g., patient in which the embryo may be implanted), and/or communicated to a patient, clinician, user of the image capturing device, etc.

In some variations, the real-time communication link may be provided by an application executed on a computing device communicably coupled to the image capturing device. The application may cause a display on the computing device to display a capture button, such that in response to a user selecting the capture button, the image capturing device captures one or more single images of the embryo.

Furthermore, in some variations, the method may include performing one or more quality control measures on the single image, such as determining whether the single image depicts an embryo (e.g., via a third convolutional neural network), and/or determining the probability that the embryo in the single image is a single blastocyst. Furthermore, in some variations, the method may include generating the viability score for the embryo in response to determining that the single image depicts an embryo. Additionally or alternatively, in some variations the method may include providing an alert to a user of the image capturing device in response to determining that the single image does not depict an embryo.

Additionally or alternatively, the method may further include predicting, via a fourth convolutional neural network, whether the embryo is euploid or aneuploid. This predicting may, in some variations, also depend at least in part on data associated with a subject (e.g., age, day of biopsy, etc.). The method may include generating a ploidy outcome based on whether the embryo is euploid or aneuploid, and updating at least the fourth convolutional neural network based at least in part on the ploidy outcome and the data.

The method may be used to predict viability of an embryo that has not been frozen, and/or viability of an embryo that has been frozen and thawed. For example, in some variations the embryo has been frozen and thawed, and the method may include receiving the single image of the embryo post-thaw, and determining viability of the embryo post-thaw via the second convolutional neural network. Determining viability of the embryo post-thaw may include classifying the single image into either a first class indicating that the embryo has survived post-thaw, or a second class indicating that the embryo has reduced viability (e.g., lower level of viability, has not survived, etc.) post-thaw. In some variations, the method may be used to predict viability of an embryo that is to undergo biopsy and/or freezing. For example, the method may include receiving the single image of the of the embryo prior to biopsy and/or freezing, and determining viability of the embryo prior to biopsy and/or freezing.

Additionally or alternatively, the method may include receiving a plurality of single images where each single image depicts a respective embryo of a plurality of embryos, generating a viability score for each embryo of the plurality of embryos bay classifying each single image via at least one convolutional network, and ranking embryos based on the viability scores for the plurality of embryos. Furthermore, in some variations, the method may include displaying the plurality of single images on a display according to the ranking of the plurality of embryos, and/or displaying the viability scores for the plurality of embryos.

In some variations, the single images that are classified via the at least one convolutional neural network are not part of a time series of images. In some variations, some of the plurality of images may originate from different image capturing devices (e.g., different instances of image capturing devices and/or different types of image capturing devices).

As described above, the viability score may, for example, represent predicted likelihood of the embryo reaching clinical pregnancy (e.g., the likelihood of the embryo reaching clinical pregnancy may be associated with an outcome of a fetal cardiac activity). In some variations, the viability score may be at least in part on data associated with a patient, such as age, body mass index, day of image capture, and donor status. Once generated, the viability score may be stored in a database associated with a patient (e.g., patient in which the embryo may be implanted), and/or communicated to a patient, clinician, user of the image capturing device, etc.

Additionally or alternatively, the method may further include predicting, via a fourth convolutional neural network, whether the embryo is euploid or aneuploid. This predicting may, in some variations, also depend at least in part on data associated with a subject (e.g., age, day of biopsy, etc.). The method may include generating a ploidy outcome based on whether the embryo is euploid or aneuploid, and updating at least the fourth convolutional neural network based at least in part on the ploidy outcome and the data.

As described above, the method may be used to predict viability of an embryo that has not been frozen, and/or viability of an embryo that has been frozen and thawed. For example, in some variations the embryo has been frozen and thawed, and the method may include receiving the single image of the embryo post-thaw, and determining viability of the embryo post-thaw via the second convolutional neural network. Determining viability of the embryo post-thaw may include classifying the single image into either a first class indicating that the embryo has survived post-thaw, or a second class indicating that the embryo has not survived post-thaw.

Generally, in some variations, the method may utilize at least one convolutional neural network trained at least in part with specialized training data. For example, a method for predicting viability of an embryo may include receiving a single image of the embryo captured with an image capturing device, and generating a viability score for each embryo by classifying each single image via at least one convolutional neural network, where the at least one convolutional neural network may, for example, be configured to generate a viability score for an embryo may be trained based on training data comprising a plurality of single images of embryos captured with a plurality of image capturing devices. Additionally or alternatively, the at least one convolutional neural network may be trained based at least in part by balancing a prevalence of outcome associated with each respective image capturing device. For example, the prevalence of outcome may include a corresponding bias representing a percentage of positive pregnancy outcomes associated with each respective image capturing device). In some variations, the single image that is classified is not part of a time series of images. As described above, the viability score may, for example, represent predicted likelihood of the embryo reaching clinical pregnancy (e.g., the likelihood of the embryo reaching clinical pregnancy may be associated with an outcome of a fetal cardiac activity). In some variations, the viability score may be at least in part on data associated with a patient, such as age, body mass index, day of image capture, and donor status. Once generated, the viability score may be stored in a database associated with a patient (e.g., patient in which the embryo may be implanted), and/or communicated to a patient, clinician, user of the image capturing device, etc.

As another example, in some variations, a method for predicting viability of an embryo may include receiving a single image of the embryo, and generating a viability score for each embryo by classifying each single image via at least one convolutional neural network, where the at least one convolutional neural network may be trained based at least in part on training data including a plurality of augmented images of a plurality of embryos. The augmented images may, for example, include rotated, flipped, scaled, and/or varied (e.g., having changes in contrast, brightness, saturation, etc.) images of the plurality of embryos. In some variations, the single image that is classified is not part of a time series of images. As described above, the viability score may, for example, represent predicted likelihood of the embryo reaching clinical pregnancy (e.g., the likelihood of the embryo reaching clinical pregnancy may be associated with an outcome of a fetal cardiac activity). In some variations, the viability score may be at least in part on data associated with a patient, such as age, body mass index, day of image capture, and donor status. Once generated, the viability score may be stored in a database associated with a patient (e.g., patient in which the embryo may be implanted), and/or communicated to a patient, clinician, user of the image capturing device, etc.

### BRIEF DESCRIPTION OF THE DRAWINGS

FIG. 1 illustrates an exemplary variation of a system for evaluating embryo viability.

FIG. 2 is a flow diagram illustrating an exemplary variation of a method for evaluating embryo viability using artificial intelligence.

FIGS. 3A-3H illustrates exemplary variations of a graphical user interface (GUI) that may be part of a plug and play software rendered on a display of a computing device to capture images of embryos.

FIG. 4 is a flow diagram illustrating an exemplary variation of a method for evaluating embryo viability using a series of convolutional neural networks.

FIG. 5 is an exemplary variation of implementing a convolutional neural network on an input image of an embryo for image cropping and image segmentation.

FIG. 6 is an exemplary variation of implementing a convolutional neural network for performing quality control.

FIG. 7 illustrates an exemplary deployment of a convolutional neural network performing quality control.

FIG. 8 is an exemplary variation of implementing a convolutional neural network for image classification and score generation.

FIG. 9 illustrates an exemplary variation of a GUI that may be part of a plug and play software rendered on a display of a computing device to display an overall viability score for an embryo.

FIG. 10 illustrates an exemplary variation of a GUI that may be part of a plug and play software rendered on a display of a computing device to display images of embryos in the order in which they are ranked.

FIG. 11 illustrate examples of augmented images following the application of random transformations to the images.

FIG. 12 illustrates an overview of characteristics within a training dataset including images of over 2000 transferred embryos with pregnancy outcomes from seven different clinics.

FIG. 13 illustrates a receiver operating characteristic curve for fresh-embryo transfers using the technology described herein compared to Gardner grading system.

FIG. 14 illustrates an exemplary variation of an image of an aneuploid embryo.

FIG. 15 illustrates an overview of characteristics within a training dataset including images of over 2000 transferred embryos with ploidy status from seven different clinics.

FIG. 16 illustrate post-thaw viability assessment results from a single site.

FIGS. 17A and 17B illustrate receiver operating characteristic (ROC) curve for example embryo transfers using CNNs described herein compared to the manual Gardner grading system.

FIG. 18A illustrates example embryo images that are top-ranked by the technology described herein.

FIG. 18B illustrates example embryo images that are lowest-ranked by the technology described herein.

FIG. 19A illustrates integrated gradients and occlusion sensitivity for example embryo images that were scored high by the technology described herein.

FIG. 19B illustrates integrated gradients and occlusion sensitivity for example embryo images that were scored low by the technology described herein.

FIG. 20 illustrates that the scores assigned by the technology described herein based on example embryo images relate to the observed pregnancy rate.

FIGS. 21A-21D illustrate example images and data from a controlled experiment to depict the biases introduced by unique optical signature of images from two different image capturing devices of two different clinics.

FIG. 22 illustrates a table that illustrates exemplary balancing training data based on prevalence of outcome for different clinics.

FIGS. 23A and 23B illustrate data from a controlled experiment to depict the biases introduced by the presence of micropipettes in an image.

### DETAILED DESCRIPTION

Non-limiting examples of various aspects and variations of the invention are described herein and illustrated in the accompanying drawings.

In vitro fertilization (IVF) is a complex reproductive assisted technology that involves fertilization of the eggs outside the body in a laboratory setting. The fertilized embryos are cultured in a laboratory dish (e.g., Petri dish) and are transferred to the uterus post-fertilization. Typically, embryos start showing a clear differentiation between the inner cell mass that forms the fetus and the trophectoderm structures that forms the placenta nearly five to six days after fertilization. This stage is referred to as the blastocyst stage. Around the blastocyst stage, the embryo outgrows the Zona Pellucida membrane surrounding the embryo in preparation for “hatching.” An embryo must reach the blastocyst stage and hatch before it can implant in the lining of the uterus. Therefore, extending embryo culture until an embryo reaches blastocyst stage gives embryologists longer to observe and assess the viability of the embryo. Furthermore, less competent embryos arrest their development prior to the blastocyst stage. Accordingly, embryos that typically progress to the blastocyst stage are a select cohort of embryos that have a greater potential to form a pregnancy.

Embryos that reach the blastocyst stage are evaluated before they are transferred in order to prioritize which embryo is to be transferred first. Traditionally, embryos are manually graded by embryologists using the Gardner or Society for Assisted Reproductive Technology (SART) grading systems. These systems require an embryologist to manually inspect an embryo under the microscope and assess three components of its morphology: the degree of blastocyst expansion, the quality of the inner cell mass, and the quality of the trophectoderm. Grades are assigned to each component in order to generate a final alphanumeric grade. However, manual grading can be complex, and it may be difficult to assign absolute grades. For instance, numeric grade may be assigned in ascending order to: very early blastocyst (having 50-75 cells), expanded blastocyst (having 100-125 cells), hatching blastocyst, and hatched blastocyst, each of which represent the degree of the blastocyst expansion. For example, a grade “4” may represent an expanded blastocyst while a grade “5” may represent a hatching blastocyst, and a grade “6” may represent a hatched blastocyst.

However, the quality of the inner cell mass and the quality of the trophectoderm at each of these stages may complicate the scoring system. For example, alphabetical grades may be assigned to represent both the quality of inner cell mass and the quality of trophectoderm. So, a grade “AA” may represent good quality inner cell mass and good quality trophectoderm. However, a grade “AB” may represent good quality inner cell mass and lower quality trophectoderm. Accordingly, a grade “4AA” may represent an expanded blastocyst with good quality inner cell mass and good quality trophectoderm. That said, it may be possible that an expanded blastocyst has top-quality inner cell mass and trophectoderm. Similarly, it may be possible that a hatching blastocyst (e.g., blastocyst in the process of hatching from the Zona Pellucida) has a slightly lower quality trophectoderm than the expanded blastocyst. In such situations, it is difficult to determine, for example, whether a 4AA embryo (representing an expanded blastocyst with top-quality inner cell mass and trophectoderm) should be considered less viable than a 5AB embryo (representing a hatching blastocyst with slightly lower quality trophectoderm). Therefore, embryologists make such decisions using intuition. It may be possible that different embryologists select different embryos, thereby making it challenging to standardize the selection process.

Throughout the years, there have been a few technologies that have been introduced with the goal of improving embryo selection. One of those technologies is time-lapse imaging. Using time-lapse imaging, a microscope may capture a sequence of images of an embryo in a periodic manner. More specifically, a sequence of microscopic images of an embryo may be captured at regular intervals (e.g., 5-20 minute intervals). The idea is to observe cellular dynamics and the behavior of cells by analyzing the periodic sequence of images captured over time. For example, measurements of events such as cell division timing, multinucleation, and reverse cleavage may be taken by observing the periodic sequence of embryo images. These measurements may be used to select an embryo for transfer. Although this provides for a somewhat more standardized approach for embryo selection process in comparison to manual grading, time-lapse imaging requires specialized time-lapse imaging systems that tend to be expensive. Not all existing microscopes can accommodate time-lapse imaging. Accordingly, time-lapse imaging technology may be hardware-driven. That is, without specialized instrumentation this technology is difficult to implement. Additionally, time-lapse imaging may require the embryos to be cultured in specialized petri dishes. Loading and unloading embryos from such specialized petri dishes may take longer time, thereby increasing the risk of damaging the embryos. The high costs of such instrumentation and other required changes to already existing workflow (e.g., using specialized petri dishes) in clinics and labs have made it challenging for time-lapse imaging to gain widespread clinical adoption.

Another technology that has been introduced more recently with the goal of improving embryo selection is preimplantation genetic testing for aneuploidy (PGT-A). PGT-A may involve performing a biopsy of the trophectoderm, then sequencing the biopsy to determine if the embryo has the correct number of chromosomes. Although this may eliminate aneuploid embryos (which lead to unsuccessful pregnancy outcomes) for transfer, it does not sufficiently characterize viability of euploid embryos, as not all euploid embryos may lead to a successful outcome (e.g., successful pregnancy). Studies have shown that within a cohort of euploid embryos, those with higher quality morphology have a higher likelihood of a successful outcome. Therefore, even with a PGT-A cycle, euploid embryos may need to be graded in order to identify appropriate embryos for transfer.

In contrast to existing technologies, the technology described herein provides a data-driven standardized approach of evaluating embryos that is easy to adopt and cost-effective. For example, the technology described herein may be hardware agnostic. A plug and play software that may be compatible with all imaging devices, microscopes, microscopic imaging devices, and/or the like (collectively referred to herein as “image capturing device”) may enable the image capturing device to capture images of the embryos in real-time. Accordingly, the technology may be adopted by any clinic or lab with already existing hardware (e.g., microscopes) without any additional hardware installation and/or cost burden.

The technology described herein may implement deep learning to score embryos according to their likelihood of reaching clinical pregnancy. For example, the technology may implement a series of one or more convolutional neural networks to analyze and classify an image of an embryo. The series of convolutional neural networks may also improve the accuracy of scoring an embryo. In some variations, a first convolutional neural network may be trained for segmenting and cropping the embryo in the image. A second convolutional neural network may be trained to perform quality control. A third convolutional neural network may be trained to perform image classification and scoring. As discussed above, the technology described herein may be hardware agnostic. Therefore, the convolutional neural networks described herein may be trained to accommodate any type of image capturing device and fit into the existing workflows of all clinics and labs while improving the accuracy of evaluating embryo viability.

In some variations, to enable the technology described herein to be compatible with a wide range of image capturing devices, the convolutional neural networks may be trained with images from different image capturing devices (e.g., different microscopes). Images from different image capturing devices may have different optics and different resolution. Because of this, when convolutional neural networks are trained with images that have different optics and different resolution, it may be possible that a bias is introduced for images from a specific image capturing device in comparison to some other image capturing device. To overcome this, the technology described herein augments the training data, as further described herein. For example, the training data may include images that may be randomly flipped, rotated, scaled and/or varied (e.g., changing brightness, contrast, and/or saturation) in order to accommodate for different optics and different resolutions.

As another example, to accommodate for even minor differences in image capturing devices (e.g., minor differences in microscopes) used across different clinics, the convolutional neural networks may be trained by balancing a prevalence of outcome for each clinic and/or image capturing device. For example, if the training data from Clinic A has 60% positive pregnancy outcomes, while the training data for the remaining clinics each have only 40% positive pregnancy outcomes, it may be likely that the convolutional neural network may learn to apply a positive bias (e.g., higher scores) for all images from Clinic A. This in turn may lead to suboptimal analysis of embryos on a per-site basis. Therefore, the technology described herein may re-sample the training data so that every clinic and/or every image capturing device has the same ratio of positive-to-negative images so as to balance the prevalence of outcome for each clinic and/or every image capturing device.

The technology described herein may identify and mitigate other biases in a similar manner (e.g., by balancing a prevalence of outcome). For instance, some captured images of an embryo may include an image of a micropipette (e.g., embryo holding micropipette) holding the embryo. The presence of micropipettes in images that may be used as training data may introduce a bias. For example, if the training data includes images with micropipettes and images without micropipettes, it may be likely that the convolutional neural network may learn to apply either a positive bias (e.g., higher scores) or negative bias (e.g., lower scores) for all images with micropipettes. The convolutional neural network may for example, focus almost exclusively on the micropipette in the images rather than the embryo to classify and score the image. This in turn may lead to suboptimal analysis of embryos that may be held by micropipettes during imaging. The technology described herein may re-sample the training data so that images with micropipettes may have the same ratio of positive-to-negative (i.e., ratio of positive pregnancy training images to negative pregnancy training images) images so as to balance the prevalence of outcome. In a similar manner, biases introduced based on the stage of the blastocyst (e.g., early blastocyst, expanding blastocyst, hatching blastocyst, hatched blastocyst, etc.) in the images may also be identified and mitigated by balancing a prevalence of outcome.

Optionally, to further improve the accuracy of prediction, the technology described herein may include patient data such as age, body mass index, donor status, and/or the like. For example, the age of patient may significantly impact the outcome of transfer despite the viability of the embryo. Therefore, incorporating patient data improves the accuracy of evaluating embryo viability. Additionally or alternatively, the technology described herein may include results from genetic test results such as prenatal genetic testing, parental genetic testing, etc., to further improve accuracy of prediction.

Furthermore, the technology described herein may analyze, classify, and score a single image of the embryo. This is a significant difference from existing time-lapse imaging technologies that analyze a time-series of embryo images collectively in order to score the embryo. In contrast to analysis of time-series images, even if multiple images (e.g., not necessarily in time-series) of the embryo are captured (such as at different focal planes and/or rotations), the technology described herein analyzes each image individually in order to produce an overall score for the embryo. For example, each individual image may be classified and scored. An average of the score across all the images may be the final score of the embryo representing the viability of the embryo. Alternatively, each individual image may be classified and scored. A median and/or a mode of the score across all the images may be the final score of the embryo representing the viability of the embryo. This may improve the accuracy of assigning a score to an embryo. For example, even if one image of the embryo is not captured well (e.g., due to selection of focal plane by the embryologist, variations in lighting, etc.), the overall score assigned to the embryo may not be significantly impacted since every image may be classified and scored individually.

Additionally, the plug and play software may enable each of the individual images to be analyzed in real-time. For example, the plug and play software may enable an embryologist to capture images of multiple embryos in real-time. These images may be analyzed and scored in real-time. The plug and play software may then display the overall viability score of the embryo in real-time. In some variations, the images may also be ranked based on the overall viability score of the embryo in that image in real-time. The plug and play software may then display the images in the order in which they are ranked. This is in contrast to existing technologies that do not display images of embryos in the order in which they are ranked. Accordingly, the most viable embryo may be displayed first, making it faster to spot and select the most viable embryo for transfer.

In addition to the above, the present technology may perform aneuploidy prediction. Additionally and/or alternatively, the present technology may provide assessments of embryos that have been frozen and thawed in order to determine if an embryo has survived the freeze-thaw process.

### System Overview

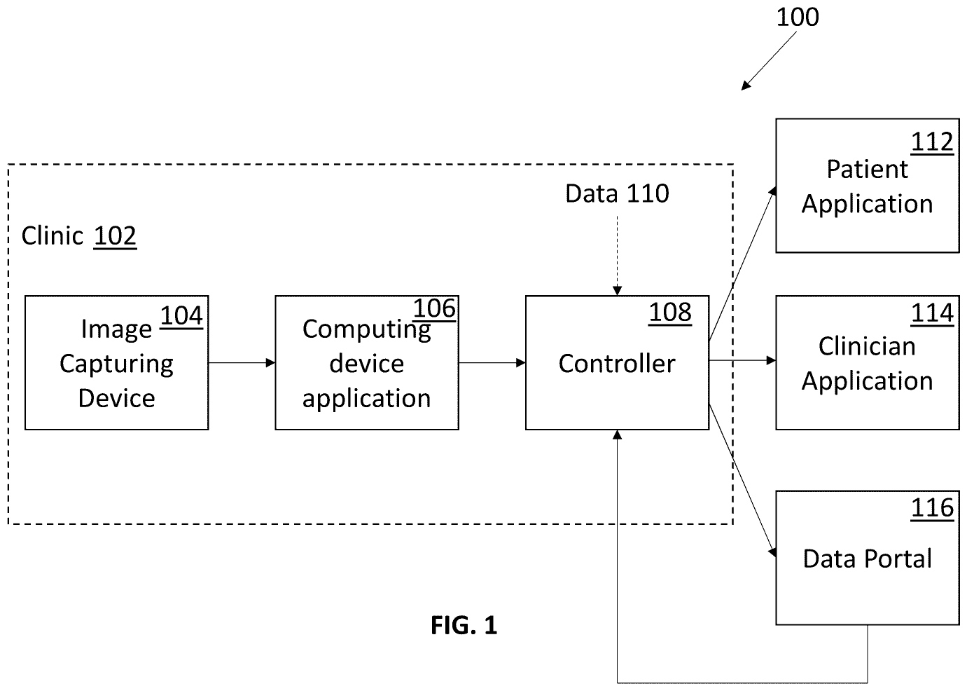

FIG. 1 illustrates an overview of an exemplary variation of a system 100 for evaluating embryo viability. The system 100 may be adopted by any clinic and/or lab 102 (referred to as “clinic” herein) with existing hardware such as an image capturing device 104. The image capturing device 104 may capture one or more images of embryos. An application 106 executed on a computing device in the clinic 102 may provide a real-time communication link between the image capturing device 104 and a controller 108. The controller 108 may use artificial intelligence to analyze the images of embryos in order to evaluate embryo viability. In some variations, the controller 108 may optionally incorporate data 110 to improve the accuracy of the evaluation. The controller 108 may score embryos in each individual image. The controller 108 may evaluate the viability of an embryo based on the score assigned to the embryo in each image. The overall viability score of the embryo may be used to rank images of the embryo. The overall viability score and the order in which the images are ranked may be transmitted to a patient application 112, clinician application 114, and a data portal 116. Each of the patient application 112, clinician application 114, and the data portal 116 may display images in the order in which they are ranked.

As discussed above, any clinic 102 may adopt the system 100 into their existing workflows. Clinic 102 may be, for example, any lab, fertility center, or clinic providing IVF treatments. Clinic 102 may include the infrastructure to culture embryos. For instance, clinic 102 may include crucial equipment needed for assisted reproductive technologies such as incubators, micromanipulator systems, medical refrigerator, freezing machines, petri dishes, test-tubes, four-well culture dishes, pipettes, embryo transfer catheters, needles, etc. Additionally, clinic 102 may provide a stable, non-toxic, pathogen free environment for culturing embryos.

An existing image capturing device 104 in the clinic 102 may capture one or more images of embryos. The image capturing device 104 may have any suitable optics and any suitable resolution. The image capturing device 104 may be a microscope, a microscopic imaging device, or any other suitable imaging device capable of capturing images of embryos. For instance, the image capturing device 104 may be any suitable microscope such as a brightfield microscope, a darkfield microscope, an inverted microscope, a phase-contrast microscope, a fluorescence microscope, a confocal microscope, an electron microscope, etc. Additionally or alternatively, the image capturing device 104 may be any suitable device operably coupled to a microscope camera capable of capturing digital images of embryos. For example, the image capturing device 104 may include a microscope camera that is operably coupled to handheld devices (e.g., computer tablet, smartphone, etc.), laptops, desktop computers, etc. In yet another alternative variation, the image capturing device 104 may be any suitable computing device (e.g., computer tablet, smartphone, laptop, and/or the like) running a microscope application capable of capturing images of embryos.

An application software 106 (referred to herein as “application”) executed on a computing device in the clinic 102 may enable the image capturing device 104 to capture one or more images of embryos. Some non-limiting examples of the computing device include computers (e.g., desktops, personal computers, laptops etc.), tablets and e-readers (e.g., Apple iPad®, Samsung Galaxy® Tab, Microsoft Surface®, Amazon Kindle®, etc.), mobile devices and smart phones (e.g., Apple iPhone®, Samsung Galaxy®, Google Pixel®, etc.), etc.

In some variations, the application 106 (e.g., web apps, desktop apps, mobile apps, etc.) may be pre-installed on the computing device. Alternatively, the application 106 may be rendered on the computing device in any suitable way. For example, in some variations, the application 106 (e.g., web apps, desktop apps, mobile apps, etc.) may be downloaded on the computing device from a digital distribution platform such as app store or application store (e.g., Chrome® web store, Apple® web store, etc.). Additionally or alternatively, the computing device may render a web browser (e.g., Google®, Mozilla®, Safari®, Internet Explorer®, etc.) on the computing device. The web browser may include browser extensions, browser plug-ins, etc. that may render the application 106 on the computing device. In yet another alternative variation, the browser extensions, browser plug-ins, etc. may include installation instructions to install the application 106 on the computing device.

The application 106 may be a plug and play software that may be compatible with any type of computing device. Additionally, the application 106 may be compatible with any type of image capturing device 104. In some variations, the application 106 may include a live viewer software that may display images of the embryos as seen through the image capturing device 104. For example, traditionally, an image capturing device 104 such as a microscope has been used to view embryos. However, with the live viewer software (included in the application 106), the computing device may display images (e.g., two-dimensional images) of embryos as seen through the image capturing device 104. A user (e.g., embryologist) may view images of embryos on a display of the computing device executing the application 106. The application 106 may enable the user to select an image that the user would like to capture. The application 106 may transmit instructions to the image capturing device 104 to capture the selected image. In some variations, the application 106 may perform a quality check before transmitting instructions to the image capturing device 104 to capture a selected image. For instance, the application 106 may analyze the selected image to determine whether the properties (e.g., resolution, brightness, etc.) of the selected image would enable further analysis. In response to meeting the quality check, the application 106 may transmit instructions to the image capturing device 104 to capture the selected image. Alternatively, the application 106 may first transmit instructions to the image capturing device 104 to capture the selected image. Once the selected image is captured, the application 106 may analyze the captured image to determine whether the properties (e.g., resolution, brightness, etc.) of the captured image would enable further analysis.

In order to enable a user to select one or more images for capture, the application 106 may render a widget (e.g., a capture button) when the application 106 is executed on the computing device. The widget may be designed so that a user may interact with the widget. The widget may be pressed or clicked (e.g., via a touchscreen or a controller such as a mouse). When a user wants to select an image for capture, the user may press or click on the widget. In response to the pressing or clicking, the image capturing device 104 may capture that specific image of the embryo. The user may choose to capture multiple images by pressing or clicking the widget repeatedly. In some variations, the widget (e.g., capture button) may be a standalone button in any suitable shape (e.g., in the form of a circle, elliptical, rectangle, etc.). In some variations, an object-oriented programming language such as C++ may be used to design and execute the application 106.

As discussed above, the application 106 may provide a real-time communication link between the image capturing device 104 and a controller 108. Accordingly, captured images of embryos may be transmitted in real-time to the controller 108 via the application 106. In some variations, the controller 108 may include one or more servers and/or one or more processors running on a cloud platform (e.g., Microsoft Azure®, Amazon® web services, IBM® cloud computing, etc.). The server(s) and/or processor(s) may be any suitable processing device configured to run and/or execute a set of instructions or code, and may include one or more data processors, image processors, graphics processing units, digital signal processors, and/or central processing units. The server(s) and/or processor(s) may be, for example, a general purpose processor, a Field Programmable Gate Array (FPGA), an Application Specific Integrated Circuit (ASIC), and/or the like.

In some variations, the controller 108 may be included in the computing device on which the application 106 may be executed (e.g., to locally perform one or more processes described herein). Alternatively, the controller 108 may be separate and operably coupled to the computing device on which the application 106 may be executed, either locally (e.g., controller 108 disposed in the clinic 102) or remotely (e.g., as part of a cloud-based platform). In some variations, controllers 108 may include a processor (e.g., CPU). The processor may be any suitable processing device configured to run and/or execute a set of instructions or code, and may include one or more data processors, image processors, graphics processing units, physics processing units, digital signal processors, and/or central processing units. The processor may be, for example, a general purpose processor, a Field Programmable Gate Array (FPGA), an Application Specific Integrated Circuit (ASIC), and/or the like. The processor may be configured to run and/or execute application processes and/or other modules, processes and/or functions associated with the system and/or a network associated therewith. The underlying device technologies may be provided in a variety of component types (e.g., MOSFET technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, and/or the like.

The controller 108 may use artificial intelligence to evaluate viability of embryos. For example, controller 108 may implement one or more convolutional neural networks to analyze and classify captured images. More specifically, the convolutional neural network(s) may analyze and classify each captured image in order to evaluate embryo viability.

It should be readily apparent that, although the user may choose to capture multiple images of a specific embryo, these images may not necessarily be time-lapse images (e.g., in time-series). For example, in time-lapse imaging, two or more images of an embryo are captured in a series at periodic or intermittent time intervals (e.g., time intervals of 5-20 minutes). To capture time-lapse images, a time-lapse microscope may be required. In contrast, as discussed above, the system 100 is compatible with any type of image capturing device 104. Accordingly, the system 100 may not necessarily capture images in a series at periodic time-intervals. Although, this may be one possible variation of the system 100, the system 100 described herein may capture multiple images in any suitable manner (owing to its compatibility with various types of image capturing devices). For example, a second image of an embryo may be captured 3 seconds after a first image of the embryo is captured. However, the third image of the embryo may be captured 5 seconds after the second image and a fourth image of the embryo may be captured 2 seconds after the third image. Accordingly, even if multiple images of an embryo may be captured and analyzed, these images may not be time-lapse images. In an exemplary variation, multiple images (e.g., at least two successive images) of an embryo may be captured within a time interval of about 60 seconds or less. For instance, two or more successive images of embryo may be captured within a time interval that ranges between about 1 second and 60 seconds, between about 5 seconds and 60 seconds, between about 10 seconds and about 60 seconds, between about 20 seconds and about 60 seconds, between about 30 seconds and about 60 seconds, between about 1 second and 30 seconds, between about 1 second and about 20 seconds, between about 1 second and about 10 seconds, or between about 1 second and about 5 seconds. Additionally, unlike time-lapse images that capture a set number of images in series at periodic time intervals for every embryo, system 100 may capture different number of images for every embryo. For instance, time-lapse images may capture three images in series at period intervals for each embryo in order to determine the viability of the embryo. In contrast, system 100 may capture three images of a first embryo and two images of a second embryo. The system 100 may determine the viability of the first embryo from the three images and the viability of the second embryo from the two images. Accordingly, the number of images captured for the embryos may be different for different embryos.

The convolutional neural network(s) may analyze and classify each captured image individually in order to generate an overall viability score of the embryo. For example, if the application 106 captures three images of an embryo on day 5 using the image capturing device 104, each of the three images may be evaluated individually and separately by the convolutional neural network(s). For the purposes of discussion herein, unless explicitly suggested otherwise the terms “an image,” “each image,” “the image,” “a captured image,” “each captured image,” “the captured image,” “a separate image,” and “an individual image” may be considered as a single individual image. Evaluation of each image may generate a respective score for the embryo that may be associated with a respective image of the three images. The overall score of the embryo may be a function of each individual score associated with each individual image. For instance, the overall score of the embryo may be an average of the three individual scores associated with the three individual images. Alternatively, in another example, the overall score of the embryo may be a median of the three individual scores associated with the three individual images. In some variations, the overall viability score of the embryo generated by the convolutional neural network(s) may indicate a likelihood of the embryo resulting in clinical pregnancy if transferred (e.g., a likelihood of successful outcome).

The convolutional neural network(s) may include a series of convolutional neural networks to perform one or more of the following: (1) image segmentation and image cropping; (2) quality control; (3) image classification; and (4) optionally to incorporate data 110 to generate an accurate overall embryo viability score. The series of convolutional neural networks may be implemented by the server(s) and/or processor(s). For instance, the server(s) and/or processor(s) may include software code to implement each of the convolutional neural network. More specifically, each convolutional neural network may be included in the software code as a separate module. When the server(s) and/or processor(s) execute the software code, the individual modules may generate instructions to perform (1) image segmentation and image cropping; (2) quality control; (3) image classification; or (4) incorporate data 110. Additionally or alternatively, the software code may include calls to separate modules implementing a respective convolutional neural network. A call to a specific module may redirect the processing performed by server(s) and/or processor(s) to implement the specific convolutional neural network included within that module. In some variations, two or more convolutional neural networks may be implemented simultaneously by the server(s) and/or processor(s). Alternatively, the convolutional neural networks may be implemented in series one after another. In some variations, the convolutional neural networks may be implemented and/or trained using PyTorch or Tensorflow.

As discussed above, in order to refine the overall viability score assigned to embryos, in some variations, the system 100 may incorporate data 110 (e.g., patient data and/or embryo data). Data 110 may include, for example, patient data associated with one or more patients such as patient's age, patient's body mass index, and/or the like, and/or embryo data. For example, patient data may include the age of a patient undergoing IVF treatment. This may have an impact on the pregnancy outcome. It may be possible that two similarly scored embryos may lead to different pregnancy outcomes depending on the age of the patient. Additionally or alternatively, patient data may include data relating to one or more donors associated with the patient, such as donor's age, donor's status, donor's body mass index, and/or the like. For example, patient data may include body mass index of a patient. This may play a factor contributing to health of the embryo. As another example, patient data may include an indication of whether a patient is a first-time patient. If not, patient data may additionally include whether embryos associated with the patient have had a previous successful outcome. In this manner, patient data may enable reproductive endocrinologists, embryologists, and/or clinicians to personalize IVF treatments. In some variations, the patient data may include data relating to one or more genetic testing results such as prenatal genetic testing result, embryo level genetic testing result, parental genetic testing result, etc. Additionally or alternatively, in some variations, data 110 may include embryo data, such as, for example, genetic testing results regarding aneuploidy, disposition to disease, potential future traits, sex, etc. Furthermore, in some variations, other embryo-specific data, such as the day the image of the embryo was captured, the day the embryo is transferred, and/or the like may be used to further improve the accuracy of the prediction.

For the purposes of discussion herein, data 110 may, for example, refer to: (1) data associated with one or more patients and/or one or more donors that may include the description, content, values of records, a combination thereof, and/or the like; and/or (2) metadata providing context for the said data. For example, data 110 may include one or both the data and metadata associated with patient records and/or donor records. Data 110 may be extracted from reliable electronic medical records. For instance, the system 100 may access one or more third party databases that may include electronic medical records, such as eIVF™ patient portal, Artisan™ fertility portal, Babysentry™ management system, EPIC™ patient portal, IDEAS™ from Mellowood Medical, etc., or any suitable electronic medical record management software.

As discussed above, the convolutional neural network(s) implemented on the controller 108 may score embryos (e.g., generate an overall viability score) according to their likelihood of reaching clinical pregnancy, and in some variations, may also rank images (e.g., rank each image based on overall viability score of embryo in that image). The respective overall viability scores and order in which the images are ranked may be transmitted to patient application 112, clinician application 114, and data portal 116. In some variations, the patient application 112 may be executed on a computing device (e.g., computers, tablets, e-readers, smartphones, mobile devices, and/or the like) associated with a patient. The patient may access the patient application 112 on the computing device in order to view the overall viability scores for embryos and ranks of images. The patient application 112 may display the images of the embryos in the order of their ranks. Therefore, a most viable embryo may appear first on the display. This makes it easy for the patient to identify the most viable embryo and make crucial decisions related to the IVF treatment.

In a similar manner, the clinician application 114 may be executed on a computing device (e.g., computers, tablets, e-readers, smartphones, mobile devices, and/or the like) associated with a clinician (e.g., embryologist, reproductive endocrinologist, etc.). The clinician may access the clinician application 112 on the computing device in order to view the overall viability scores of embryos and ranks of images. The clinician application 112 may display the embryos in the order of their ranks. In some variations, the clinician application 114 may be same as application 106 described above. For instance, the application 106 that enables the image capturing device 104 to capture images of embryos may also display overall viability embryo scores and order in which the images are ranked after the embryos are evaluated by the controller 108. Equivalently, in addition to displaying the overall viability scores and the order in which images are ranked, the clinician application 114 may enable the image capturing device 104 to capture images of embryos. Alternatively, the clinician application 114 may be different from the application 106 described above. For instance, the clinician application 114 may be executed on a computing device that may be different from the computing device that executes application 106.

The data portal 116 may be a data collection software that may store the scores (e.g., score associated with each individual image and the overall viability score for an embryo) and/or ranks that were generated by the controller 108. The collected data may be analyzed at the data portal 116 for further improving the accuracy of the system 100. For example, the collected data may be processed and provided as additional training data to the convolutional neural network(s) implemented by the controller 108. Accordingly, the convolutional neural network(s) may become more intelligent further enhancing the accuracy of predicting embryo viability. In some variations, the data portal 116 may be connected to one or more databases. The database(s) may store the scores (e.g., score associated with each individual image and the overall viability score for an embryo), rank, patient data, and/or other related data related to the embryo. In some variations, the data portal 116 may be connected to a memory that stores these database(s). Alternatively, the data portal 116 may be connected to a remote server that may store these database(s). In some variations, results from the controller 108 may be transmitted to one or more third party databases that may include electronic medical records, such as eIVF™ patient portal, Artisan™ fertility portal, Babysentry™ management system, EPIC™ patient portal, IDEAS™ from Mellowood Medical, etc. These results may include overall viability score of embryos, rank, etc.

### Exemplary Method for Evaluating Embryo Viability

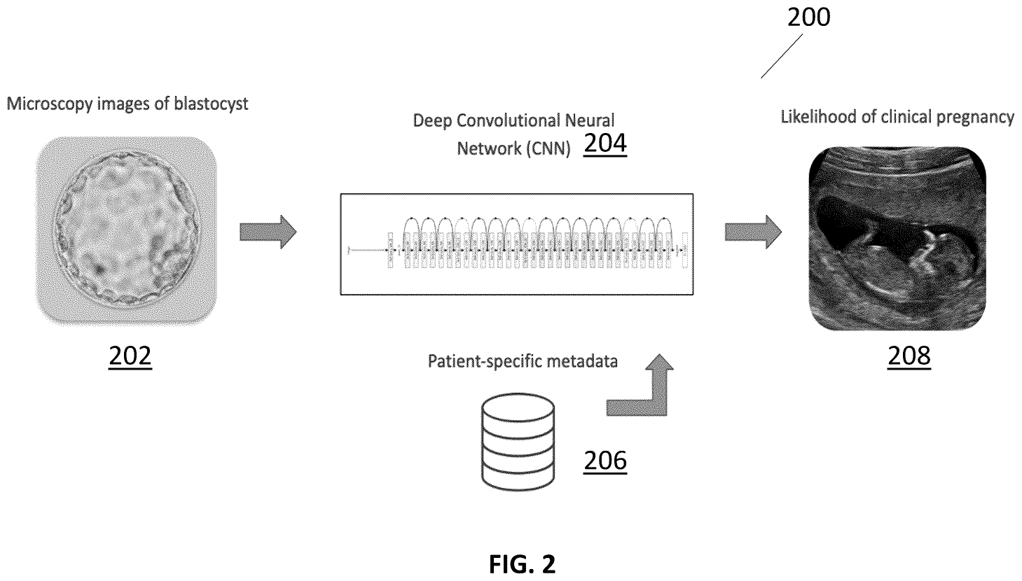

FIG. 2 is a flow diagram illustrating an exemplary variation of a high-level overview of a method 200 for evaluating embryo viability using artificial intelligence. In some variations, the method 200 may be implemented using a system such as system 100 described in FIG. 1. At 202, the method 200 may include capturing one or more images of an embryo. In particular, the images of the embryo may be images captured once the embryo reaches a blastocyst stage (e.g., day 5, day 6, and/or day 7 post-fertilization). That is, the captured images may be microscopy images of a blastocyst. In some variations, the embryo may be evaluated at any of different stages of a freeze-thaw process, such prior to freezing, or after freezing and thawing. Additionally or alternatively, the embryo may be evaluated prior to biopsy. In other variations, the embryo is not intended to undergo biopsy or freezing.

At 204, each image may be individually analyzed and classified by at least one deep convolutional neural network (D-CNN) in real-time. The D-CNN may be implemented on a controller such as controller 108 in FIG. 1. For example, the D-CNN may evaluate a single image in real-time. If multiple images of an embryo (e.g., multiple images of a blastocyst) are captured, each image may be analyzed and classified individually to generate an overall viability score for the embryo. The D-CNN may also rank the images based on the overall viability score of the embryo in the images. In some variations, the D-CNN may incorporate patient data 206 (e.g., patient-specific metadata) such as age, body mass index, donor status, etc., to improve the accuracy of the overall score assigned to the embryo.

At 208, the method 200 may predict the likelihood of an embryo reaching clinical pregnancy based on the overall viability score of the embryo generated by the D-CNN. In some variations, the overall viability score indicating the likelihood of clinical pregnancy (e.g., successful outcome) may be displayed on one or more displays. In some variations, the rank of the images (e.g., rank of the image determined by the D-CNN) may also be displayed. For example, images of embryos may be displayed in the order of their ranks. In this manner, a clinician (e.g., embryologist, reproductive endocrinologists, clinician, etc.) may select an embryo for transfer in real-time in consultation with a patient based on the overall viability score of the embryos and the ranks of the images generated by the D-CNN.

### Capturing an Image of an Embryo

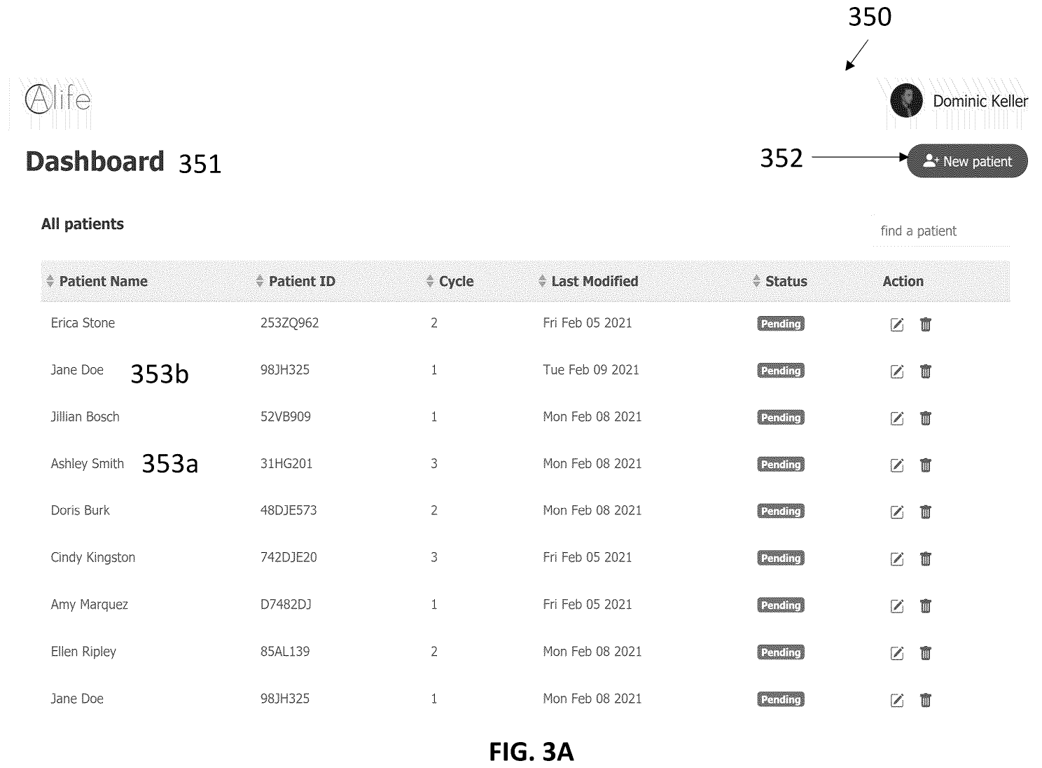

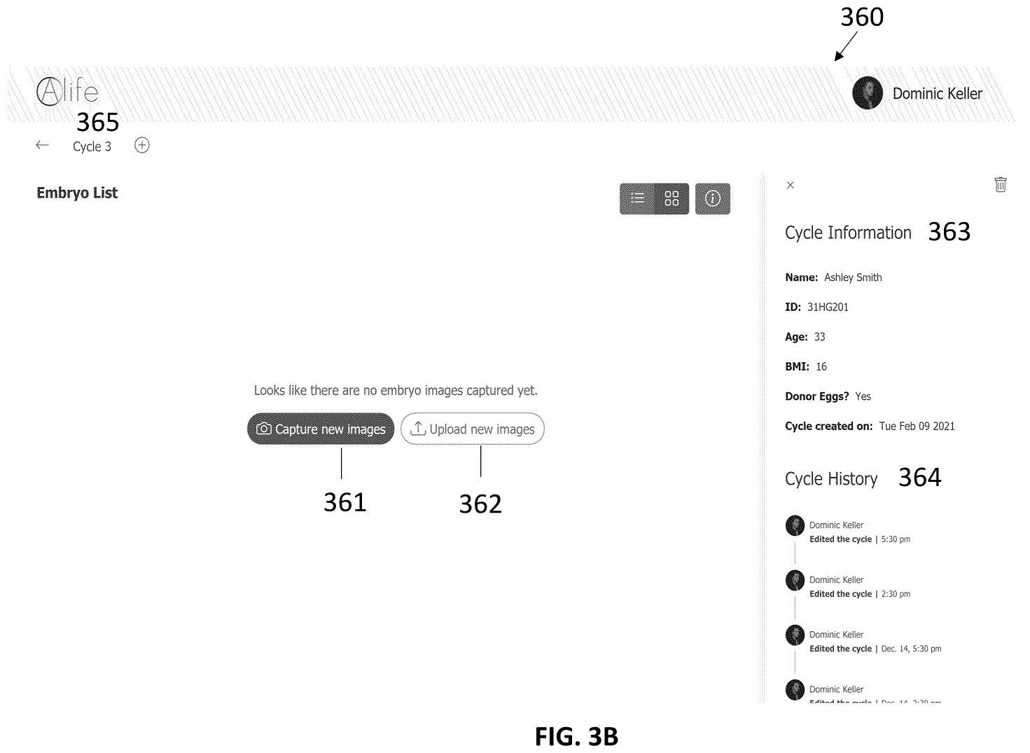

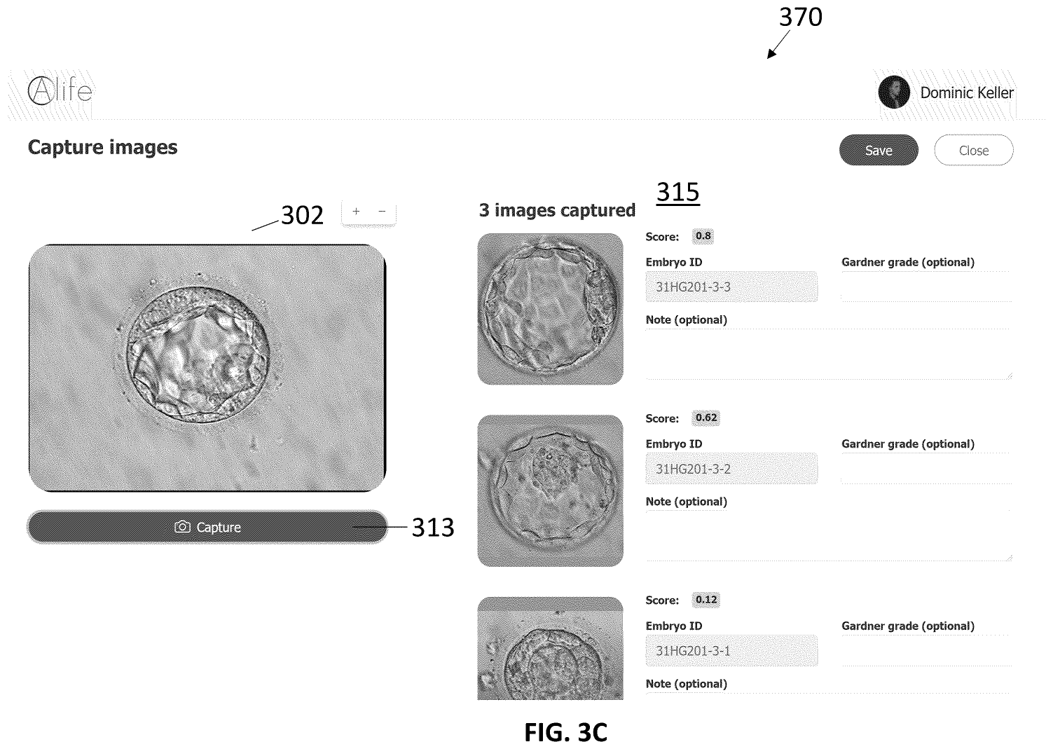

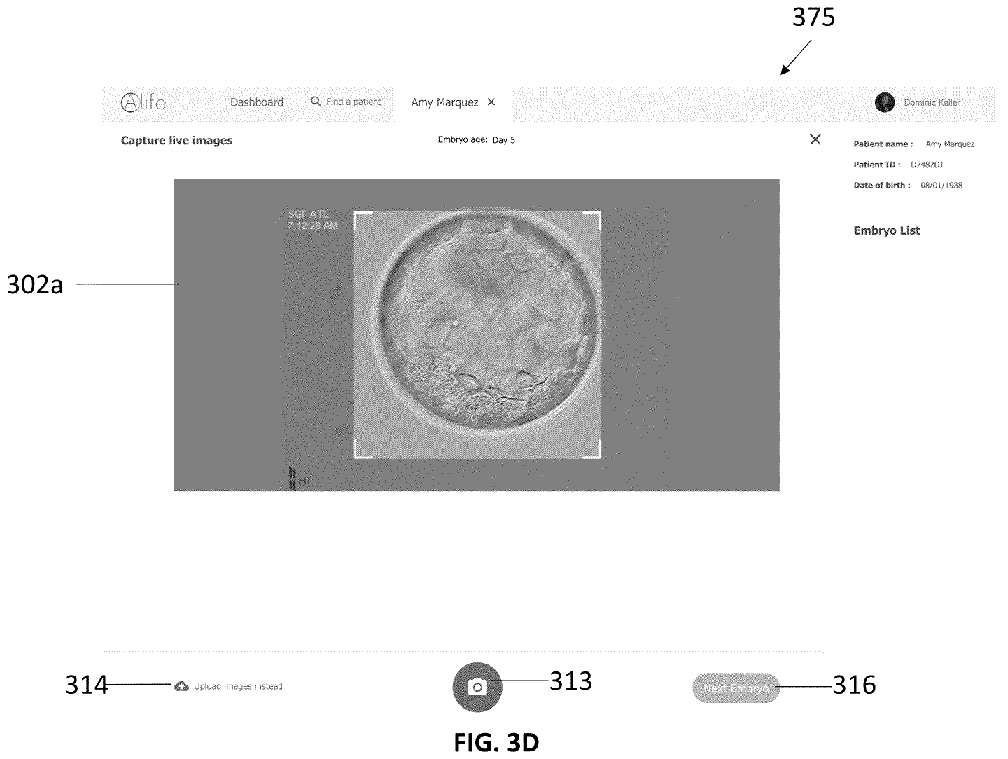

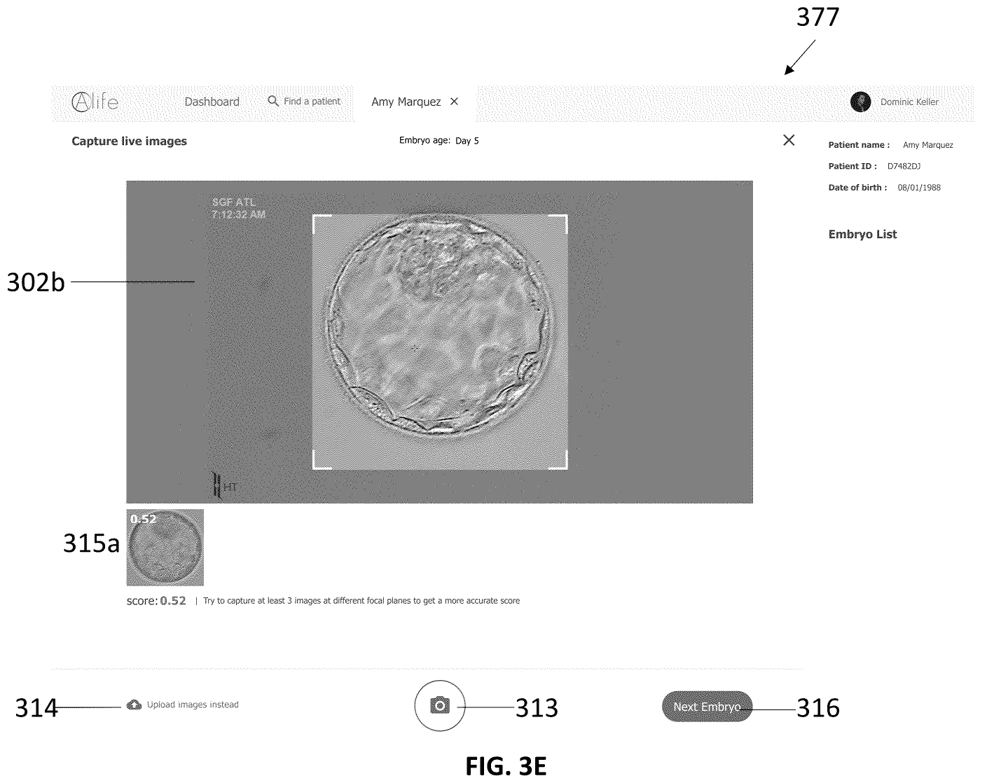

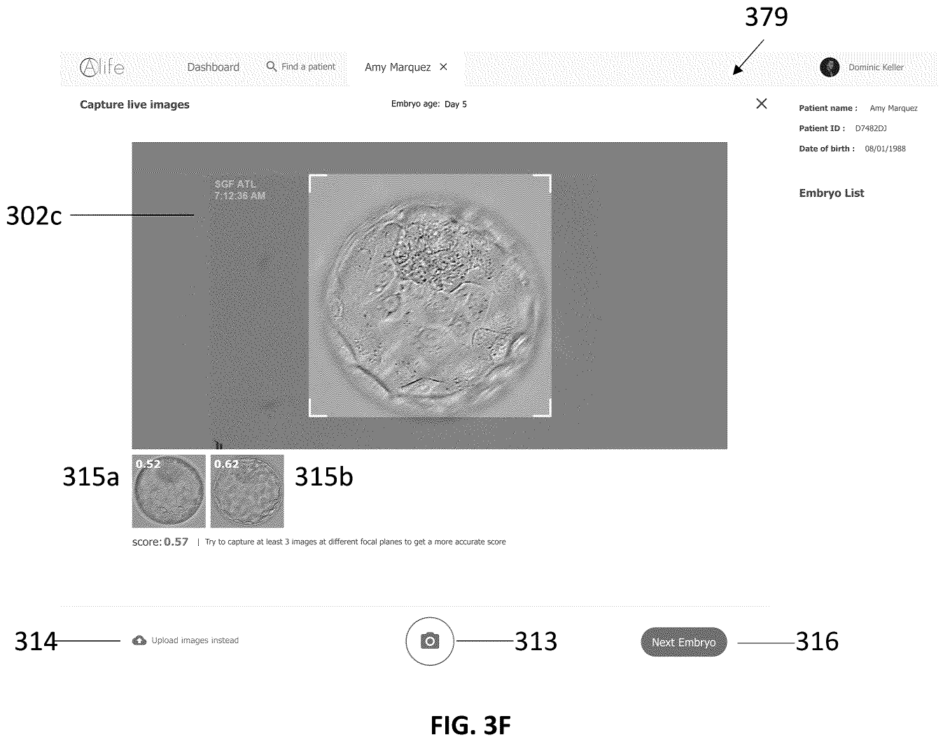

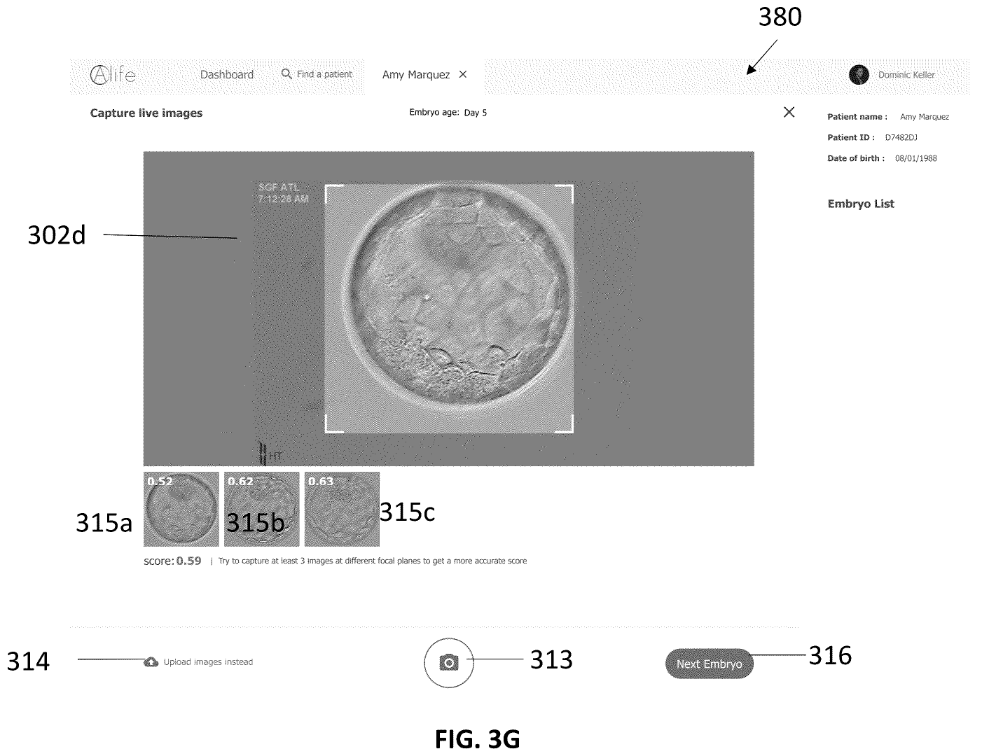

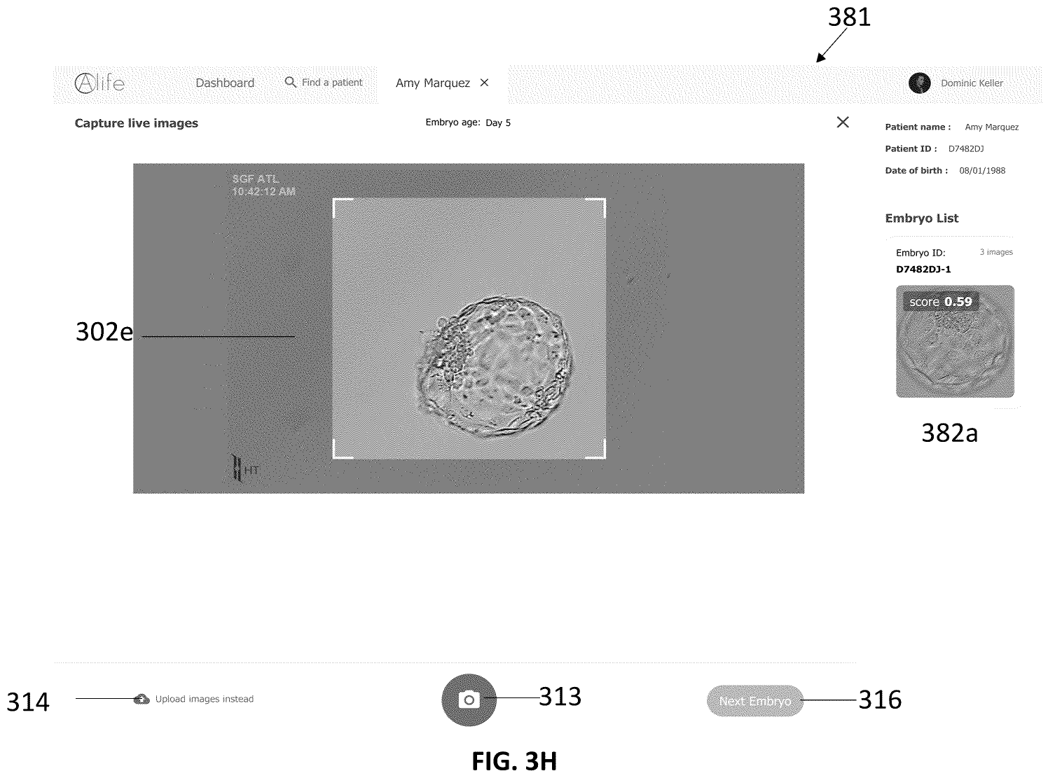

As discussed above, the technology disclosed herein may be adopted by any clinic (e.g., clinic 102 in FIG. 1) with already existing hardware. The technology disclosed herein may be compatible with any type of image capturing device (e.g., image capturing device 104 in FIG. 1) and may be adopted by a clinic without having to make changes to their existing workflow. Accordingly, a plug and play software such as application 106 in FIG. 1 may enable a clinician to capture one or more images of an embryo using an existing image capturing device. FIGS. 3A-3H illustrate exemplary variations of a graphical user interface (GUI) that may be a part of the plug and play software rendered on a display of a computing device to capture images of embryos. In some variations, the computing device may be pre-installed with the application. Alternatively, the application may be downloaded on the computing device from a web store. In yet another alternative variation, rendering a web browser with specific browser extensions may render the application on the computing device.

The application may cause the computing device to display various functionalities. For example, the application may cause computing device to manage image capture and other actions associated with patients. As an illustrative example, as shown in FIG. 3A, the application may render display 350 that may include a dashboard 351. The dashboard 351 may represent an initial step in a workflow towards determining embryo viability. In some variations, the dashboard 352 may include some information of various patients (e.g., “Ashley Smith” 353a, “Jane Doe” 353b etc.). For example, the dashboard 351 may include patient ID (e.g., represented as “Patient ID” in FIG. 3A) of a patient, number of transfer cycles associated with a patient (e.g., represented as “Cycle” in FIG. 3A), the status of embryo transfer associated with a patient (e.g., represented as “Status” in FIG. 3A), etc.

In some variations, the display 350 may include a widget designed for user interaction such as widget “New patient” 352. For instance, by clicking and/or pressing on the “New patient” 352, a user may add a patient not already listed on the dashboard 351 including information (e.g., patient ID, number of cycles, status, etc.) associated with the patient. In some variations, the information may be inputted manually by a user interacting with the display 350. Alternatively, the information may be extracted from a database (e.g., a third-party electronic medical record database). For instance, the application may interact with an electronic medical record database to access and extract information related to the patient. Although, the widget “New patient” 352 in FIG. 3A may be illustrated as a button that is elliptical in shape, it should be readily apparent that any suitable widget may be provided. For example, the “New patient” may be a circular shaped widget, a triangular shaped widget, etc.

In order to access a specific patient's information, the user may press and/or click on the row containing information of the patient. For instance, by pressing and/or clicking on the row containing information related to “Ashley Smith” 353a, the application may transition from display 350 in FIG. 3A to another display (e.g., display 360 in FIG. 3B) that may include further specific information associated with “Ashley Smith” 353a.

Display 360 in FIG. 3B may include additional information associated with the patient's (e.g., “Ashley Smith” 353a) cycle (e.g., represented as “Cycle information” 363). This information may include patient ID, age, body mass index, whether the eggs belong to a donor, etc. Display 360 may further include past data and/or history associated with the current cycle and/or previous cycle (e.g., represented as “Cycle History” 364). For instance, this may include embryo scores associated with a past cycle and the corresponding outcome of the cycles. If embryo images relating to the current cycle does not exist (e.g., application does not have any embryo images for “Ashley Smith” 353a in the currently cycle), display 360 may include an option to capture new images or upload new images (e.g., from a pre-existing database). For example, display 360 may include one or more widgets designed for user interaction such as widgets “Capture new images” 361, “Upload new images” 362, etc. By clicking and/or pressing on the “Capture new images” 361, a user may use the application to instruct an image capturing device to capture images of an embryo. More particularly, the application may transition from display 360 in FIG. 3B to display 370 in FIG. 3C. By clicking and/or pressing on the “Upload new images” 362, the user may upload already captured images of an embryo to the application. Although the widget “Capture new images” 361 and the widget “Upload new images” 362 in FIG. 3B may be illustrated as a button that is elliptical in shape, it should be readily apparent that any suitable widget may be provided.

The application may cause the computing device to display one or more user interface elements for facilitating capture of embryo images. For example, the application may include a live viewer software to display images of embryos as seen through an image capturing device (e.g., microscope). As an illustrative example, as shown in FIG. 3C, image 302 is the image of an embryo as seen through a microscope. A widget designed for user interaction such as a capture button 313 may enable a user to capture the image 302 via the image capturing device. For example, in FIG. 3C, the widget may be a capture button 313 including the text “Capture.” For instance, by clicking and/or pressing on the capture button 313, an image such as image 302 may be captured. In this manner, one or more images of embryos may be captured in real-time using a plug and play software. Display 370 may also include previously captured images and their respective scores. For example, as seen in FIG. 3C, image 302 may be the fourth image to be captured for a specific patient (e.g., “Ashley Smith” 353a). The other three images may be displayed under images captured 315. Some of these three images may be images of the same embryo while other images under image captures 315 may be images of different embryos. Additionally or alternatively, some of the three images may be captured on a same day while some other images of the three images may be captured on a different day. In FIG. 3C, the three images are images of different embryos. The viability score for each of the embryos is represented next to the respective images. The images 315 may include information regarding the embryo ID and the respective viability score associated with the image. Additionally, a user may enter notes relating to the images via the display 370.

FIG. 3D illustrates another variation of a display 375 with one or more user interface elements for facilitating capture of embryo images. As discussed above, the application may include a live viewer software to display images of embryos as seen through an image capturing device (e.g., microscope). In FIG. 3D, image 302a is a first image of an embryo as seen through a microscope.

Unlike FIG. 3C, the widget designed for user interaction such as a capture button 313 in FIG. 3D may include an icon (e.g., camera icon) instead of the text “Capture.” By clicking and/or pressing on the capture button 313, a first image such as image 302a may be captured. Alternatively, a user may click and/or press the widget 314 to upload images of embryos that were previously captured.

In order to capture multiple images of the same embryo, a user may click and/or press the capture button 313 multiple times. Additionally or alternatively, in response to a user clicking, pressing, and/or holding the capture button 313 for at least a predetermined duration of time, the application may capture multiple images of the embryo in succession (e.g., burst mode). Additionally or alternatively, the capture button 313 may be associated with a timer such that in response to a user clicking, pressing, and/or holding the capture button, the application may capture multiple images within an allocated or predetermined period of time set by the timer.

As discussed above, the application can capture multiple images of the same embryo. Each image may be scored individually. In some variations, one or more outlier images may be flagged, rejected and/or eliminated. For example, an image of an embryo with a viability score drastically different (e.g., differing at least by a predetermined threshold from the viability score associated with every other image of the same embryo, an average viability score of other images of the same embryo, etc.) may be automatically flagged for review (e.g., by the user), automatically rejected and excluded from characterization of the embryo but still present for viewing, and/or automatically discarded or deleted entirely. The overall viability score of the embryo may be a mathematical function of each of the individual scores (e.g., average, median, mode, etc.). As a user captures an image of the embryo, the application scores the image in real-time. In some variations, these captured images and/or scores may be displayed in real-time to the user. For example, the first image 302a captured in FIG. 3D may be displayed as an already captured image 315a in FIG. 3E. The score of the first image 302a captured in FIG. 3D may be displayed next to image 315a (e.g., same as image 302a) in FIG. 3E. In FIG. 3E, the score of the image 315a is 0.52. A second image 302b of the embryo may be viewed through the live viewer software. The second image 302b may be captured by clicking and/or pressing on the capture button 313 or by clicking and/or pressing widget 314 to upload images of the embryo that was previously captured.

The first image 302a captured in FIG. 3D and the second image 302b captured in FIG. 3E may be displayed as image 315a and image 315b in FIG. 3F respectively. The score of the first image 302a captured in FIG. 3D may be displayed next to image 315a (e.g., same as image 302a) in FIG. 3F. As seen in FIG. 3E, the score of the image 315a is 0.52. Similarly, the score of the second image 302b captured in FIG. 3E may be displayed next to image 315b (e.g., same as image 302b) in FIG. 3F. In FIG. 3F, the score of the second image 315b is 0.62. The overall viability score of the embryo may be displayed below image 315a and image 315b. For example, in FIG. 3F, the overall viability score (e.g., 0.57) is the average of the score of the image 315a (e.g., 0.51) and score of the image 315b (e.g., 0.62). In other words, a running average viability score for an embryo may be calculated in real-time based on multiple captured images of the embryo as the images are captured, and may be updated as images are captured and/or deleted. A third image 302c of the embryo may be viewed through the live viewer software. The third image 302c may be captured by clicking and/or pressing on the capture button 313 or by clicking and/or pressing widget 314 to upload images of the embryo that was previously captured.

The first image 302a captured in FIG. 3D, the second image 302b captured in FIG. 3E, and the third image 302c captured in FIG. 3F, may be displayed as image 315a, image 315b, and image 315c in FIG. 3G respectively. The score of the first image 302a captured in FIG. 3D may be displayed next to image 315a (e.g., same as image 302a) in FIG. 3G. As seen previously, the score of the image 315a is 0.52. Similarly, the score of the second image 302b captured in FIG. 3E may be displayed next to image 315b (e.g., same as image 302b) in FIG. 3F. As seen previously, the score of the image 315b is 0.62. Similarly, the score of the third image 302c captured in FIG. 3F may be displayed next to image 315c (e.g., same as image 302c) in FIG. 3G. The score of the image 315c is 0.63. The updated overall viability score of the embryo may be displayed below image 315a, image 315b, and image 315c. For example, in FIG. 3G, the overall viability score (e.g., 0.59) is the average of the score of the image 315a (e.g., 0.51), score of the image 315b (e.g., 0.62), and score of image 315c (e.g., 0.63). A fourth image 302d of the embryo may be viewed through the live viewer software. The fourth image 302d may be captured by clicking and/or pressing on the capture button 313 or by clicking and/or pressing widget 314 to upload images of the embryo that was previously captured. Although FIG. 3G illustrates a fourth image 302d that may be captured, it should be understood that any suitable number of images (e.g., 1, 2, 3, 4, 5, or more) may be captured for the embryo, displayed, and/or provide a basis for overall viability score of the embryo.

If the user chooses to capture an image of a different embryo, the user may press and/or click on the new embryo widget 316. The application may transition from display 380 in FIG. 3G to display 381 in FIG. 3H. The live viewer software may display images of another embryo (e.g., image 302e) as seen through an image capturing device (e.g., microscope). The overall viability score of the already scored embryo (e.g., embryo in FIGS. 3D-3F), may be displayed next to image 382a. For instance, image 382a may be one of the first image 302a of the previously scored embryo captured in FIG. 3D, the second image 302b of the previously scored embryo captured in FIG. 3E, or the third image 302c of the previously scored embryo captured in FIG. 3F. The overall viability score (e.g., 0.59 as seen in FIG. 3G) may be displayed next to the image 382a. A user can repeat the process as outlined in FIGS. 3D-3G in order to determine the overall viability score of the embryo seen in FIG. 3G.

### Evaluating Embryo Viability

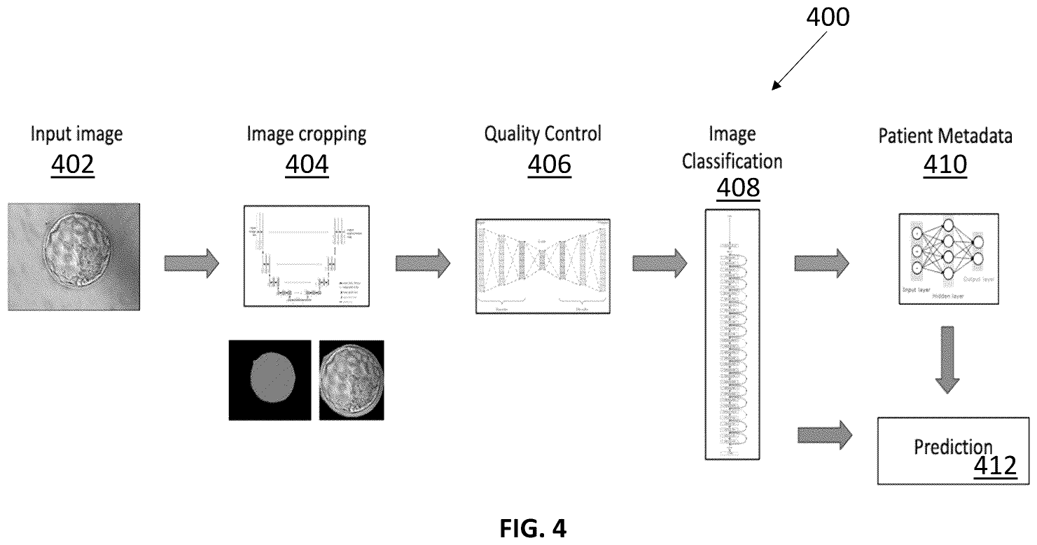

Once an image is captured, the image may be sent in real-time to a controller (e.g., controller 108 in FIG. 1) for evaluation. The controller may implement one or more convolutional neural networks (CNNs) to assess and classify the image in real-time. In some variations, the CNNs may be a series of CNNs. FIG. 4 is a flow diagram illustrating an exemplary variation of a method 400 for evaluating embryo viability using a series of CNNs.

At 402, the controller may receive an input image such as image 302 in FIG. 3 captured by a plug and play software. The input image may be analyzed by a series of CNNs each implemented to perform: (1) image segmentation and image cropping at 404; (2) optionally quality control at 406; (3) image classification at 408; and (4) optionally incorporate patient data at 410. At 412, the method 400 may predict the viability of the embryo based on the output from image classification (e.g., at 408) and optionally based on the output from incorporating patient data (e.g., at 410). In some variations, the viability of the embryo represents the likelihood (e.g., a value between 0 and 1) of the embryo reaching clinical pregnancy. In some variations, positive fetal cardiac activity or negative fetal cardiac activity may be considered as an indicator of clinical pregnancy.

CNNs typically comprise of one or more convolutional layers to extract features from an image. The convolutional layers may include filters (e.g., weight vectors) to detect specific features. The filters may be shifted stepwise across the height and the width dimensions of the input image to extract the features. The shifting of the filters (i.e., the application of filters at different spatial locations) provide translation invariance. For example, if features representing a boundary of an embryo appears at a first spatial location in one image and the same features appear at a second different spatial location in another image, then owing to the translation invariance of the CNNs, these features can be extracted from both the first spatial location and the second spatial location. Accordingly, translation invariance provides a feature space in which the encoding of the input image may have enhanced stability to visual variations. That is, even if the embryo slightly translates and/or rotates from one image to another image, the output values do not vary much.

As discussed above, a series of CNNs may be implemented to extract features and/or classify the input image. These CNNs and their architectures are further described below.

### Image Cropping

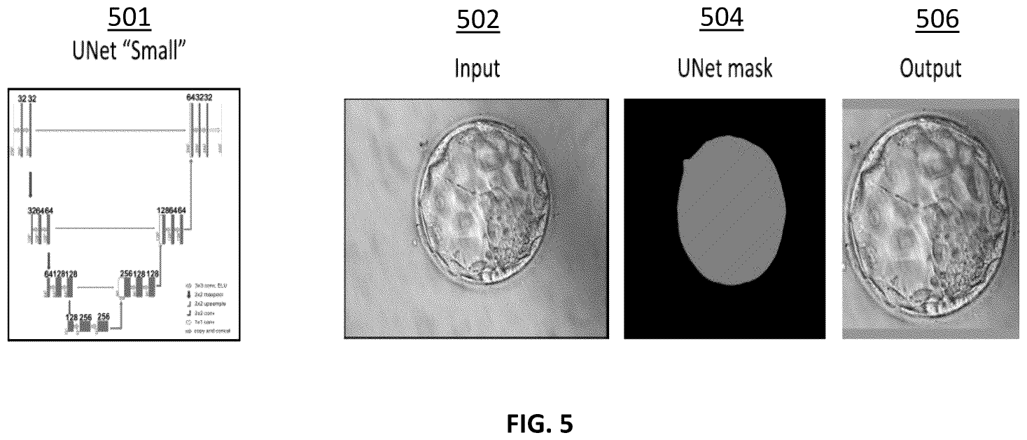

The CNN implementing image segmentation and image cropping (e.g., at 404 in FIG. 4) may take an input image of an embryo to generate an output image cropped to a boundary of the embryo. FIG. 5 is an exemplary variation of implementing a CNN on an input image of an embryo for image cropping and image segmentation. In an exemplary variation, a U-Net 501 architecture may be used for image segmentation and image cropping. U-Net 501 architecture may provide for precise and fast segmentation of embryos. The left part of the U-Net 501 architecture may include a contracting path that may produce a low-dimensional representation of the input image and a right part of the U-Net 501 architecture may include an expansive path that may up-sample the low dimensional representation to produce a segmentation map.

The trained U-Net 501 architecture may generate a U-Net mask 504 for segmentation of embryos. The U-Net mask 504 may be a ground truth binary segmentation mask. The U-Net 501 may compare an input image 502 to the U-Net mask 504 to create a square crop around the embryo in the input image. The U-Net 501 may then generate an output image 506 cropped to the boundary of the embryo. In an exemplary variation, the hyperparameters for the U-Net 501 architecture may include 40 Epochs; lr=0.0005, and batch=32.

In alternative variations, the architecture of the CNN for image cropping and image segmentation may include any suitable CNN such as Mask R-CNN, fully convolutional network (FCN), etc.

### Quality Control

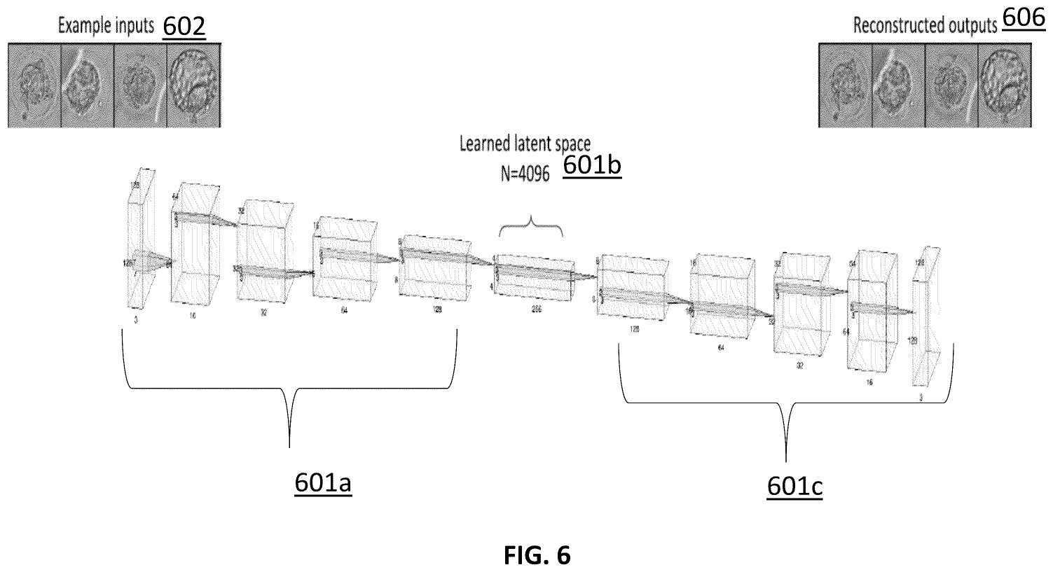

The output image (e.g., output image 506 in FIG. 5) generated by the CNN implementing image cropping and image segmentation may act as an input to the CNN performing quality control. Quality control of an image may include verifying whether an image includes an embryo and/or determining a probability that the embryo is a blastocyst. As a first step to performing such verification, since the images have already been cropped and segmented to a boundary of the embryo (e.g., using the U-Net 501 in FIG. 5), the CNN performing cropping may eliminate images that do not have a foreground result (e.g., depict no embryo). Alternatively, the CNN performing cropping may transmit an alert such as a warning message and/or a warning signal to a user (e.g., via the application 106 executing on the computing device) to eliminate images that do not have a foreground result (e.g., depict no embryo). In yet another alternative variation, the CNN performing cropping may transmit a modification (e.g., via the application 106 executing on the computing device) to change the image so as to include the embryo in the image and improve the quality of the image. This may act as a first filter. The rest of the images (i.e., the image that were not eliminated) may be verified by implementing the quality control CNN shown in FIG. 6.

FIG. 6 is an exemplary variation of implementing a CNN for performing quality control. In an exemplary variation, an autoencoder may be used for performing quality control. The architecture may include 5 layers of two-dimensional convolutions including convolutional layers, pooling layers, input layer, and output layer. For example, the 5 layers of two-dimensional convolutions may include strides, batch normalization, and ReLU activation. Layers 601a may represent the encoder, 601b may represent the latent space, and layers 601c may represent the decoder of the autoencoder. As seen in FIG. 6, the dimensionality of an image may be first reduced (e.g., encoder layers 601a). From this reduced encoding, the CNN may reconstruct the image (e.g., using decoder layers 601c). The latent space 601b may represent the compressed state of the data. The autoencoder may identify outliers in the latent space 601b. Due to the compression and the subsequent reconstruction, the model may be rid of any extraneous noise, thereby focusing on the important features. FIG. 6 shows example inputs 602 and the corresponding reconstructed outputs 606 generated by implementing the autoencoder shown in FIG. 6.

In an exemplary variation, the hyperparameters for the autoencoder may include 200 Epochs; lr=0.003, and batch=32. The learned latent space may be N=4096.

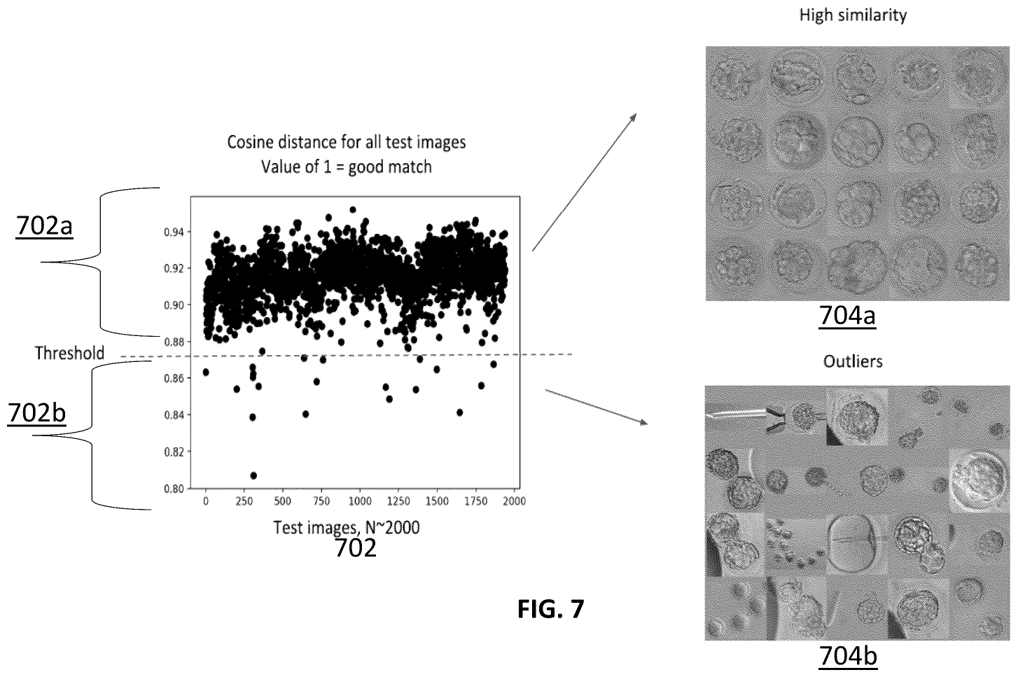

FIG. 7 illustrates an exemplary deployment of a convolutional neural network (e.g., autoencoder in FIG. 6) performing quality control. The autoencoder may extract the learned latent space for 100 random training images. The autoencoder may then take an average of the latent space. For instance, in the example provided in FIG. 7, the average produces N=2000. A latent space clustering method may be used to identify outliers. For example, a distance (e.g., cosine similarity) between a sample point in the latent space and a reference point may be computed. It may be observed that similar data points cluster together. For instance, points with cosine distances nearly equal to 1 may be similar data points that cluster together (e.g., as cluster 702a) in graph 702. The points with cosine distances lower than a threshold value (e.g., lower than 0.87 in FIG. 7) may be outliers (e.g., outliers 702b). The images of the embryos with high similarity that cluster together (e.g., corresponding to points 702a) is shown in 704a. The images of embryos that may be outliers (e.g., corresponding to points 702b) is shown in 704b. Any suitable CNN that may perform quality control as described above may be used (e.g., a regular CNN classifier).

### Image Classification

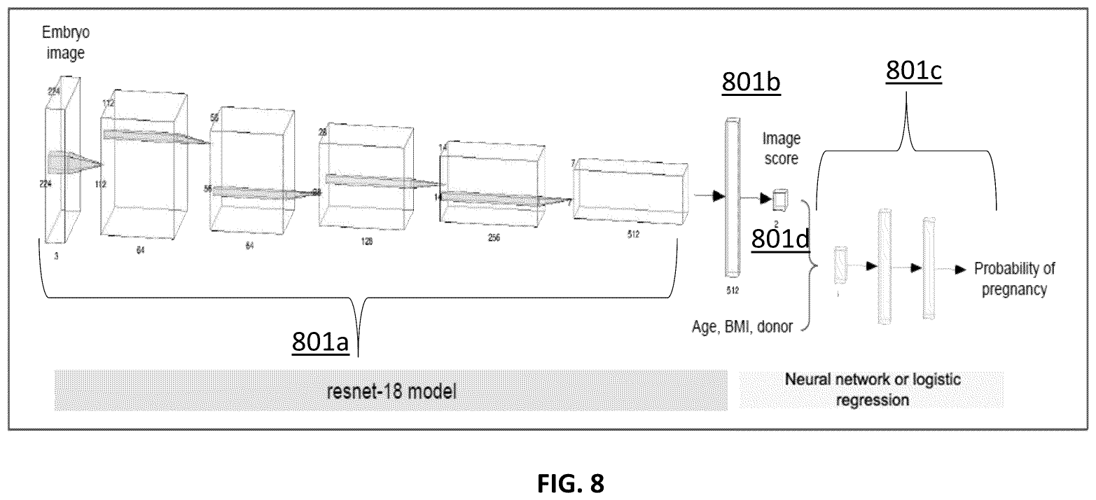

After cropping and segmenting an input image, and optionally performing quality control in some variations, the image may be classified using another CNN. FIG. 8 is an exemplary variation of implementing a CNN for image classification and score generation. The CNN may be trained to classify the image to generate an image score 801d (e.g., viability score). The image score 801d may indicate a probability that the embryo in the image will reach clinical pregnancy based on the evaluation of that image, probability that the embryo in the image will reach live birth, and/or the like.

In an exemplary variation, a resnet-18 model 801a architecture may be used with transfer learning for image classification. The resnet-18 model 801a may be a residual network that is 18 layers deep. The resnet-18 model 801a may include one or more residual blocks. Identity mappings created by residual blocks may allow the residual blocks to skip connections without affecting the residual network's performance. Transfer learning may allow the resnet-18 model 801a to transfer knowledge learned by performing similar tasks for a different dataset. That is, a resnet-18 model 801a may be a pre-trained model pre-trained on a different dataset (e.g., ImageNet). Transfer learning may be performed to fine tune the resnet-18 model 801a for some or all layers and to repurpose the resnet-18 model 801a to classify images of embryos. In some variations, a shallow architecture of resnet-18 model 801a as shown in FIG. 8 may be implemented. This may minimize the risk of overfitting and may reduce computational requirements. In alternative variations, modified versions of DenseNet, Inception, Alexnet and/or GoogLeNet may be used for image classification instead of resnet models.

In some variations, as an optional step, patient data may be incorporated in order to improve the accuracy of predicting embryo viability. In some variations, variables such as patient age, body mass index, and/or donor status may be obtained from electronic medical records. Patient data may be incorporated by concatenating each image score 801d (e.g., viability score generated for an embryo in a specific image) with the corresponding patient data and/or patient metadata. A small feedforward neural network or logistic regression model 801c may incorporate these concatenated image scores and patient data. For instance, the feedforward neural network 801c may be trained on concatenated values of image scores and patient data (further details on training the CNNs below). The feedforward neural network 801c may include layers with batch normalization, ReLU, and dropout. The feedforward neural network 801c may then generate a final score representing a likelihood of successful pregnancy for an embryo in the specific image that was cropped and classified. In other implementations, the patient data can be concatenated with the final feature vector layer in the image classification model 801a, for concurrent training on images and patient data.

If more than one image of an embryo is captured, the overall viability of the embryo may be a function of individual viability scores that may be generated for individual images. For example, the overall viability score may be a mean and/or a median of individual viability scores generated for individual images. In this manner, a series of CNNs may be used to evaluate embryo viability.

### Displaying Output

As discussed above, a plug and play software such as application 106 in FIG. 1 may capture the images of embryos in real-time. Each image may be evaluated by one or more convolutional neural networks (CNNs) in real-time. Accordingly, the CNNs may generate a score for each image in real-time. This score that is generated in real-time for a captured image may be transmitted to the application 106 so as to display the score to a clinician (e.g., embryologist, reproductive endocrinologist, etc.). In some variations, multiple images of an embryo may be captured within a span of a couple of seconds. In such variations, the CNNs may generate an individual score for each captured image in real-time. However, the overall viability score for the embryo may be a function of each individual score. For such variations, the overall viability score may be transmitted to the application 106 in real-time so as to display the overall viability score to a clinician.

The score of an embryo may be displayed in any suitable manner. For instance, the score may be displayed as a percentage indicating a likelihood of successful clinical pregnancy (e.g., 90% indicating that the embryo has a 90% chance of successful clinical pregnancy). Alternatively, the score may be displayed as a number from a numerical scale (e.g., number between 0-10 with 0 representing a least viable embryo and 10 representing a most viable embryo, number between 0-100 with 0 representing a least viable embryo and 100 representing a most viable embryo, etc.). In yet another alternative variation, the score may bucket the embryo into a letter scale (e.g., “A,” “B,” “C,” “D,” etc., with “A” representing a least viable embryo). In yet another alternative variation, the score may bucket the embryo into categories (e.g., “good,” “bad,” etc.). In yet another alternative variation, at least a portion of an image of the embryo that may be displayed may be color coded with the color representing viability of the embryo. For example, a frame or border of an image of the embryo may be color coded such that the colors may be mapped onto a numerical score. In some examples, an embryo may be bucketed into a letter scale, categories, colors, and/or the like at least in part by comparing a numerical viability score to one or more predetermined thresholds.

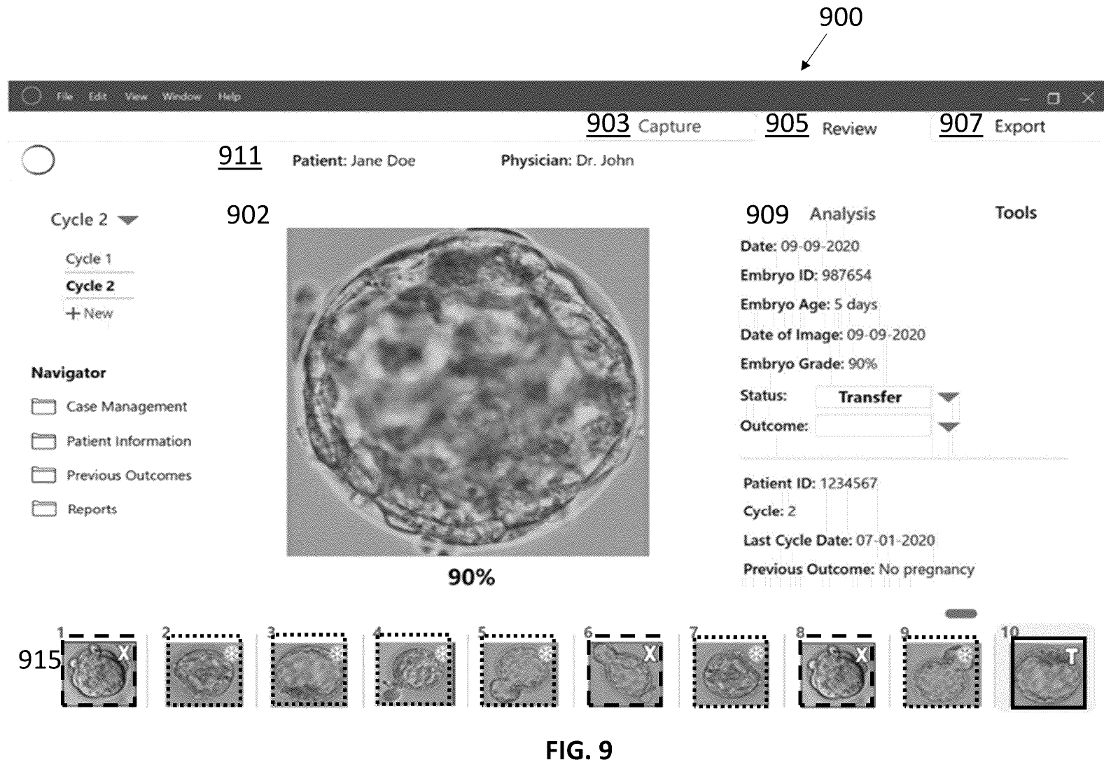

FIG. 9 illustrates an exemplary variation of a plug and play software rendered on a display 900 of a computing device to display an overall viability score for an embryo. The display 900 may include capture 903, review 905, and export 907. A user may interact with a specific option so as to cause the application to display widgets and information associated with that option. In FIG. 9, clicking on the review 905 option may cause the application to display widgets and information associated with the review 905 functionality.

FIG. 9 illustrates an instance of review 905 for the patient “Jane Doe.” In FIG. 9, image 10 may be captured in real-time and sent to a controller for analysis. The controller may classify the image and generate an overall viability score of 90% for the embryo shown in image 10 (e.g., estimated 90% likelihood of resulting in successful clinical pregnancy, if transferred to a patient). The application may cause the display 900 to display image (e.g., image 902) and the overall viability score for the embryo in the image 902. The display 900 may also include analysis 909 related to the embryo. For instance, the analysis 909 may include information such as embryo ID, number of days post-fertilization, date the image was taken, and an embryo grade (e.g., overall viability score for the embryo), etc.

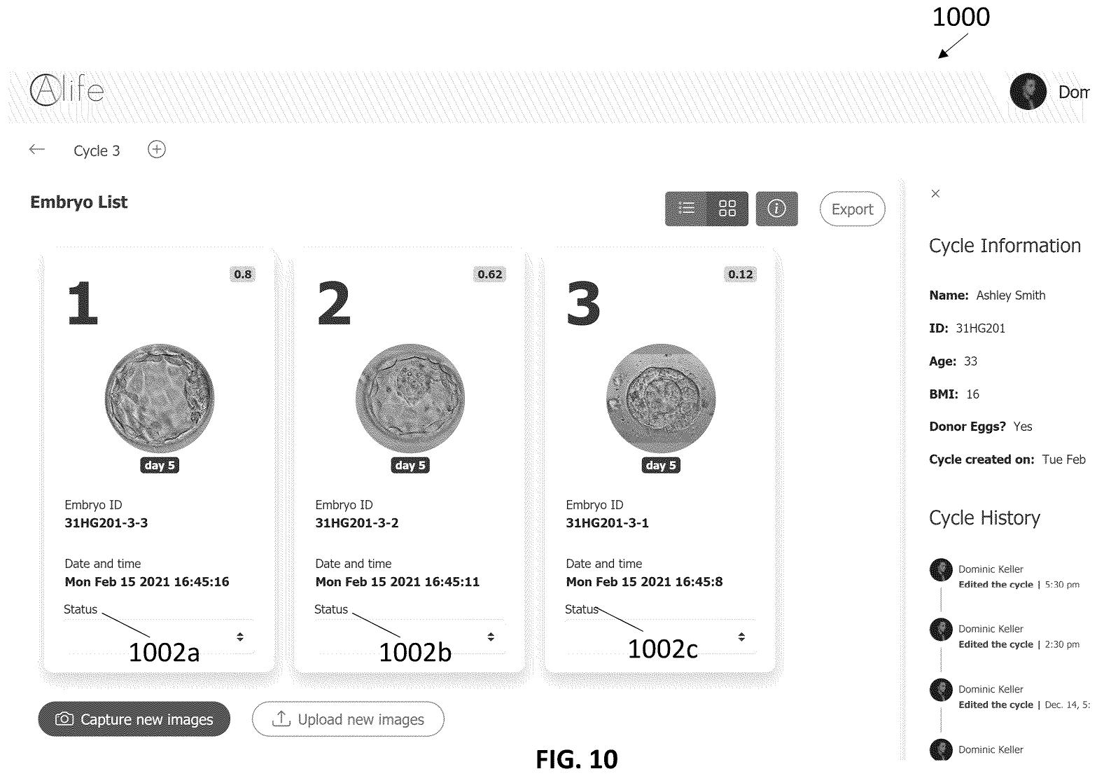

Additionally, a user (e.g., embryologist) may choose what to do with each embryo (e.g., denote embryo status for transfer, freeze, discard, etc.) based on the overall viability score of each embryo, and the embryo status may be indicated in any suitable manner (e.g., icons, text, color-coding, etc.). For example, in FIG. 9, the user may choose to discard embryos of image 1, image 6, and image 8 (indicated with an “X” on the image). The user may choose to freeze embryos of image 2, image 3, image 4, image 5, image 7, and image 9 (indicated with a snowflake icon). The user may choose to transfer embryo of image 10 (indicated with a “T”). In this manner, the user may be presented with options to decide what to do with each image, and indicate desired action for each embryo. Other variations of a user interface for facilitating these actions are described further below with respect to FIG. 10.

In addition to displaying the overall viability score (e.g., an indication of a likelihood of clinical pregnancy) for embryos in real-time, the application may also display the images in the order in which they are ranked (e.g., images with embryos having the highest likelihood of clinical pregnancy to images with embryos having the lowest likelihood of clinical pregnancy). FIG. 10 illustrates an exemplary variation of a plug and play software rendered on a display 1000 of a computing device to display images of embryos in the order in which they are ranked. In FIG. 10, the images displayed on display 1000 are images of different embryos (e.g., image with embryo ID 31HG201-3-3, image with embryo ID 31HG201-3-2, and image with 31HG201-3-1). All the three images in FIG. 10 are captured on the same day (e.g., day 5). However, the images may be captures on any suitable day (e.g., day 3, day, 4, day 6, day 7, etc.).

When the images of each embryo are sent to a controller implementing CNNs for analysis, the controller may generate an overall viability score of each embryo. For instance, the controller may individually analyze and individually score each image captured for the embryo with embryo ID 31HG201-3-3. The overall viability score of the embryo with embryo ID 31HG201-3-3 may be a function of the score of each captured image. In FIG. 10, the overall viability score of the embryo with embryo ID 31HG201-3-3 was determined to be 0.8. In a similar manner, the controller may individually analyze and individually score each image captured for the embryo with embryo ID 31HG201-3-2. The overall viability score of the embryo with embryo ID 31HG201-3-2 may be a function of the score of each captured image. In FIG. 10, the overall viability score of the embryo with embryo ID 31HG201-3-2 was determined to be 0.62. Similarly, the overall viability score of the embryo with embryo ID 31HG201-3-1 was determined to be 0.12.

The controller may then rank the embryos based on the overall viability score of the embryos in the images. For instance, in the example in FIG. 10, the controller may rank embryo with embryo ID 31HG201-3-3 and overall viability score of 0.8 as first, embryo with embryo ID 31HG201-3-2 and overall viability score of 0.62 as second, and embryo with embryo ID 31HG201-3-land overall viability score of 0.12 as third. In this manner, at least one image of each embryo may be displayed in the order in which they are ranked.

In some variations, the overall viability score of the embryo may be displayed proximate to each image of the embryo. For example, in FIG. 10, the overall viability score of 0.8 is displayed proximate to image 1. Additionally, the display 1000 may include a drop-down menu (e.g., drop-down menu 1002a for image 1, 1002b for image 2, and 1002c for image 3) below each image of the embryo with an option to select an embryo status. The user may choose to a status via the drop-down menu 1002a (e.g., “Fresh Transfer,” “Freeze,” “Freeze & Biopsy,” “Discard” etc.). The status may be indicated with text, symbols or other icons, color-coding, and/or in any suitable manner. The display 1000 may also include notes below each image displaying notes by a clinician about the embryo. The display 1000 may also indicate an embryo ID indicating the embryo that is being viewed on the display 1000.

### Training Convolutional Neural Network(s)

In order to perform a true data-driven approach to embryo assessment, the CNNs described herein may be trained on large amounts of data. The data may be collected from varied sources including a consortium of clinical partners, databases comprising microscopy images of embryos, electronic medical records, and/or the like. The collected data may include microscopy images of embryo along with electronic medical record data that may contain pregnancy outcomes for transferred embryos, Gardner grades, preimplantation genetic testing for aneuploidy (PGT-A) results for embryos that may have been biopsied and tests, and patient data. After collecting the data, the microscopy images in the collected data may be split into two groups based on their pregnancy outcome. For example, the microscopy images in the collected data may be split into positive fetal cardiac activity (FCA) representing a positive pregnancy outcome and negative fetal cardiac activity (FCA) representing a negative pregnancy outcome.

After splitting the collected microscopy images into positive FCA and negative FCA, the images in these individual groups may further be divided in any suitable ratio to form training data and testing data, such as 70% training data and 30% testing data. For example, the microscopy images with positive FCA may be split into 70% training and 30% testing. Similarly, the microscopy images with negative FCA may be split into 70% training and 30% testing. In a similar manner, in order to incorporate patient data, the embryo score for each image may be concatenated with patient data. The concatenated data may be split into 70% training data and 30% testing data.

Since the present technology is designed to be compatible with any image capturing device, the training data to train the CNNs may have to include a combination of images from different image capturing devices. Since different image capturing devices may have different optics and different resolutions, the training data may have to account for such differences.

### Augmenting Training Data

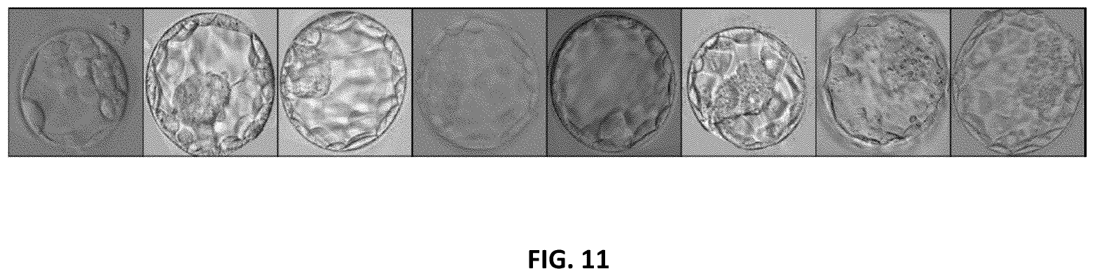

One method to account for differences in optics and resolution may be to augment the training data. Each image in the training data may be augmented on the fly. For example, one or more transformations may be applied to each image. Some non-limiting examples of random transformations include randomly flipping an image up or down, randomly flipping an image left or right, randomly scaling an image (e.g., scaling the image by −5% of the original image size), randomly rotating the image between −90 degrees and 90 degrees, randomly varying the contrast, brightness, and/or saturation of the image, a combination thereof, and/or the like. FIG. 11 illustrate an exemplary variation of examples of augmented images following the application of random transformations to one or more images of embryos. In this manner, by augmenting the training data, the CNNs may be able to account for at least some changes to optics and resolution while analyzing the images.

### Balancing Prevalence of Outcome

Despite augmenting the training data, it may be possible that the CNNs may still introduce a bias when scoring an image based on a prevalence of outcome for each clinic. For example, if the training data from one clinic has a considerably higher percentage of positive outcomes in comparison to every other clinic, the CNNs may learn to apply a positive bias for all images from that clinic. This may lead to suboptimal analysis of embryos since the embryos from the clinic with the higher positive outcome in training data may generate false positives. Similarly, if the training data has images that include micropipettes to hold embryos, the CNNs may learn to apply a positive or negative bias for all images with micropipettes.

Accordingly, to solve this problem, the training data may be re-sampled so that every clinic and/or site may have the same ratio of positive-to-negative images in each epoch of training. Similarly, the training data may be re-sampled so that images with micropipettes have the same ratio of positive-to-negative images as images without micropipettes in each epoch of training. The CNNs trained in this manner may be able to balance the prevalence of outcome and may be able to score the images with better accuracy (e.g., without introducing bias).

In this manner, by implementing a combination of augmenting the training data and balancing a prevalence of outcome, the present technology may accommodate any type of image capturing device and may fit into any existing workflow for any clinic and/or site.

### Exemplary Training Examples

In some variations, the training dataset may include images of transferred embryos with pregnancy outcomes (e.g., images of over 1000, 2000, 3000, 4000, 5000, 10,000, or more transferred embryos with pregnancy outcomes from seven different clinics). In some variations, Python and open-source framework PyTorch may be used to train CNNs. In some variations, training may be performed for 50 epochs, for example. A final model may be selected from the epoch with the highest accuracy. In some variations, a series of models may be trained using hyperparameter search to find the optimal values for parameters such as learning rate and batch size. An ensemble of 2 or more models trained with varying data sampling, hyperparameters, and/or architectures may be deployed to perform final prediction (e.g., evaluation of embryo viability).

Training a U-Net for image cropping and image segmentation: In some variations, the training data for the U-Net described above may include manual foreground labels for a few hundred raw embryo images, augmented image training data with random flip and/or rotation to create a few thousand images and masks, and images and masks square-padded and then resized, such as to 112×112.

Training an Autoencoder for quality control: In some variations, the training data for the autoencoder described above may include several thousand images from two clinics. This data may include a combination of images of frozen embryos and images of fresh embryos. As discussed above, the collected image data (e.g., collected data of images of frozen embryos and images of fresh embryos) may be divided in any suitable ratio to form training data and test data, such as 70% training data and 30% test data. In some variations, the training data may also include embryo-cropped images (e.g., images cropped by the U-Net model), resized to a suitable size such as 128×128.

Training a fully connected neural network to incorporate patient data: In some variations, the training data for a fully connected neural network described above may include embryo score for each image concatenated with patient data. This data may be divided in any suitable ratio to form training data and test data, such as 70% training and 30% testing.

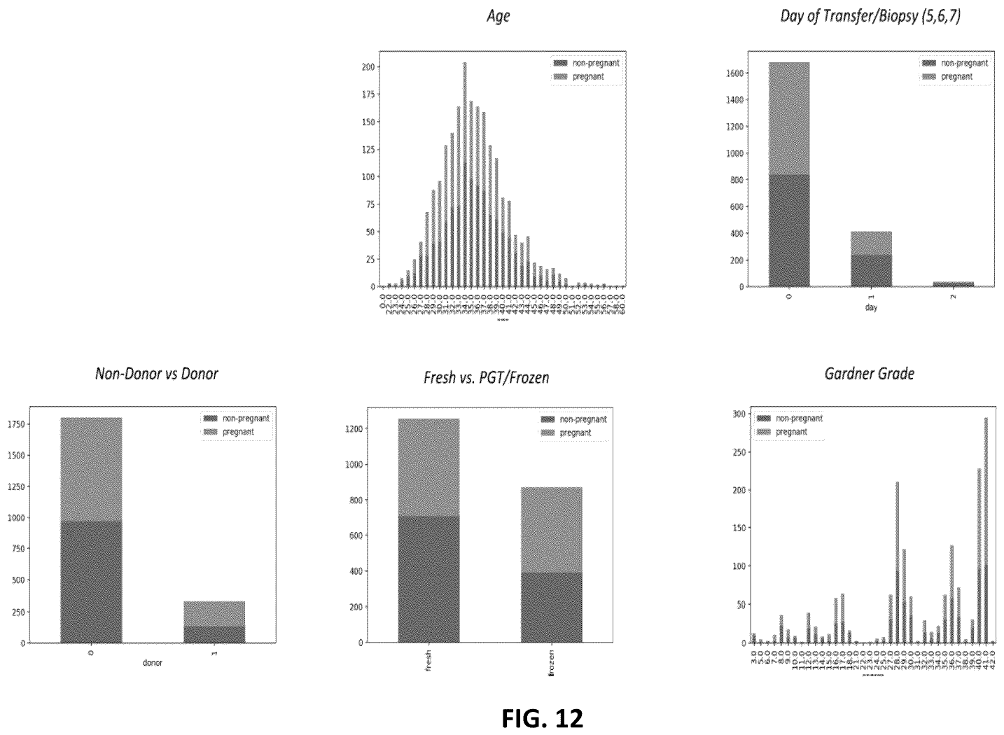

In some variations, the training dataset may include images of transferred embryos (or other suitable number) with pregnancy outcomes from seven different clinics. FIG. 12 illustrates an overview of characteristics within this training dataset. For each image of an embryo with an associated transfer outcome, information regarding patient age, day of transfer, donor status, and/or the like may be collected. In some variations, key parameters that may introduce a potential bias in the CNNs may be tracked. These parameters may include patient age, ethnicity, the day of transfer (day 5, 6 or 7), donor vs. non-donor oocytes, fresh transfers vs. frozen transfers, and Gardner grade. The plots in FIG. 12 show the distribution of the embryo dataset by outcome.

### Exemplary Performance Data

In order to evaluate a performance of the present technology (e.g., performance of the CNNs), a primary performance metric may be area under the curve (AUC). The AUC may be derived from receiver operating characteristic (ROC) curve. The AUC may be defined as a model's ability to rank instances in a binary classification problem. In this example, the AUC may measure how accurately the CNNs described herein may score an embryo with positive outcome (e.g., positive FCA) over an embryo with negative outcome (e.g., negative FCA).

In order to create a reference standard, Gardner grades were collected from IVF clinics. The alphanumeric Gardner grades (e.g. 3AA or 5AB) were mapped to a numeric score (1 through 43). The mapping was performed using an order technique that assumes that 1<2<4<5<6 for degree of blastocyst expansion and that C<B<A for both inner cell mass quality and trophectoderm quality. Accordingly, the order of grading may be:
[1, 2, . . . , 42, 43]=
[‘2CC’, ‘2BC’, ‘2CB’, ‘2BB’, ‘2BA’, ‘2AB’, ‘2AA’,
‘3CC’, ‘4CC’, ‘5CC’, ‘6CC’,
‘3CB’, ‘4CB’, ‘5CB’, ‘6CB’,
‘3BC’, ‘4BC’, ‘5BC’, ‘6BC’,
‘3CA’, ‘4CA’, ‘5CA’, ‘6CA’,
‘3AC’, ‘4AC’, ‘5AC’, ‘6AC’,
‘3BB’, ‘4BB’, ‘5BB’, ‘6BB’,
‘3BA’, ‘4BA’, ‘5BA’, ‘6BA’,
‘3AB’, ‘4AB’, ‘5AB’, ‘6AB’,
‘3AA’, ‘4AA’, ‘5AA’, ‘6AA’]

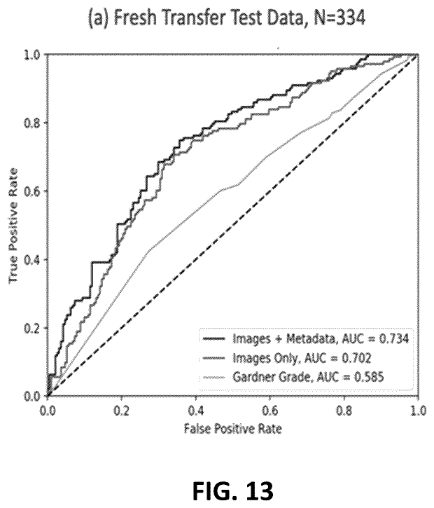

FIG. 13 illustrates receiver operating characteristic curve for fresh-embryo transfers using the technology described herein compared to Gardner grading system. The results on test data with N=334 show that the technology described herein scores embryos according to their likelihood of reaching clinical pregnancy with an AUC of 0.702 using images only, and 0.734 using a combination of images and patient data. The AUC for the manual Gardner grading system is 0.585. This represents a 15% absolute improvement using the technology described herein compared to standard of care.

### Non-Invasive Aneuploidy Prediction

In addition to scoring embryos and ranking the images based on the viability score of the embryos, present technology may also perform non-invasive aneuploidy prediction and post-thaw viability assessment. Preimplantation genetic testing for aneuploidy (PGT-A) may involve performing a biopsy of the trophectoderm which is sequenced to determine if the embryo has the correct number of chromosomes. Embryos with abnormal number of chromosomes are aneuploidy embryos while embryos with normal number of chromosomes are euploid embryos. Eliminating aneuploid embryos may eliminate embryos which may lead to unsuccessful pregnancy outcomes. Put differently, by performing PGT-A, aneuploid embryos may be eliminated from being transferred. However, existing methods of performing PGT-A prediction are invasive. In fact, there are ongoing concerns about embryo safety. More recently, the PGT-A field is starting to move towards non-invasive cell-free Deoxyribonucleic acid (DNA) testing. However, cell-free DNA testing has not been widely adopted yet. Therefore, there is an unmet need for predicting the ploidy status of an embryo non-invasively with higher accuracy.

Additionally, even if invasive PGT-A testing and/or cell-free DNA testing were to become popular, it may still be possible that more than one euploid embryo is available for transfer. Not all euploid embryos may lead to a successful outcome. Therefore, even with a PGT-A cycle, euploid embryos may need to be graded in order to prioritize an order for transfer. Additionally, grading embryos (e.g., morphological grading) in PGT-A cycles may provide adjunctive information regarding likelihood of aneuploidy and chances of leading to pregnancy.

Accordingly, the technology described herein may non-invasively predict the ploidy status of an embryo.

### Capturing an Image of an Embryo

The plug and play software described herein such as application 106 may be used to capture one or more images of embryos. The application may send images to a controller such as controller 108 in FIG. 1 in real-time. The method and/or system for capturing an image of an embryo to predict the ploidy status may be the same as the method and/or system for capturing an image of an embryo to generate a viability score (as described above).

### Predicting Ploidy Status

The controller may implement one or more deep CNNs to predict ploidy status from an image. In some variations, these CNNs may be different from the CNNs described above (i.e., CNN(s) to assess embryo viability). For example, these CNNs may be trained and modeled specifically to predict ploidy status from images of embryos. That is, instead of building CNNs to predict fetal heartbeat, the CNNs may be modeled to identify morphological features in the images that may be associated with aneuploidy.

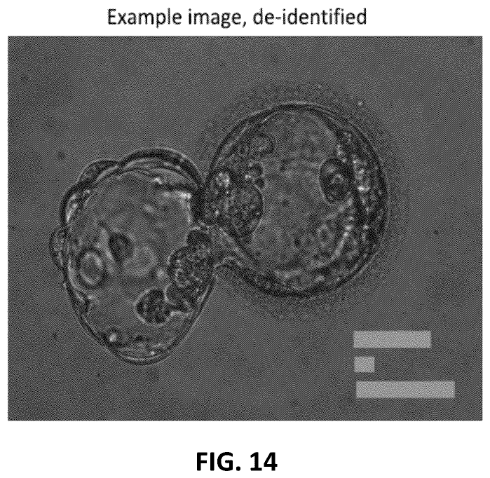

Alternatively, in addition to generating a score for an embryo, the CNNs described above may be trained to predict the ploidy status of the embryo. For example, the images captured via a plug and play software such as application 106 in FIG. 1 may be analyzed and classified by the CNNs in real-time to generate a ploidy status of the embryo. FIG. 14 illustrates an exemplary variation of an image of an aneuploid embryo. The CNNs may be trained to identify morphological features in the images that may be associated with aneuploidy.

As described herein, a series of CNNs may be implemented to analyze and classify the images. In one example, an input image may be cropped and segmented by a CNN such as a U-Net model (such as U-Net 501 in FIG. 5). The U-Net model may be trained to segment and crop the image of the embryos to a boundary of the embryo. A CNN for performing quality control such as autoencoder in FIG. 6 may be trained to identify outliers using learned latent space clustering method. The autoencoder may verify whether or not an image contains an aneuploid embryo and if it does whether the aneuploid embryo has been accurately labeled. A CNN for image classification such as resnet-18 in FIG. 8 may be trained to classify the images to generate a binary output representing ploidy status. For example, the resnet-18 may generate output “1” indicating that a embryo is not an aneuploid embryo and may generate output “0” indicating that an embryo is an aneuploid embryo. In some variations, CNNs trained for predicting ploidy status may incorporate patient data to improve the accuracy of the prediction.

In some variations, CNNs trained to identify ploidy status may incorporate patient data to improve the accuracy of the prediction. For instance, the age of a patient may be highly correlated with the ploidy status of the embryo.

### Displaying the Output

The output may be displayed in a manner similar to displaying outputs regarding viability of embryo as described above. For example, a plug and play software described herein such as application 106 may display the ploidy status for the embryo.

### Training the CNNs

The data may be collected from varied sources including a consortium of clinical partners, databases comprising microscopy images of embryos, electronic medical records, and/or the like. The collected data may include microscopy images of embryo along with electronic medical record data that may contain pregnancy outcomes for transferred embryos, preimplantation genetic testing for aneuploidy (PGT-A) results for embryos that may have been biopsied and tests, and patient data. After collecting the data, the microscopy images in the collected data may be split into two groups based on their ploidy status. For example, the microscopy images in the collected data may be split into euploid embryos and aneuploid embryos.

In some variations, the training data may include a combination of pregnancy outcome and ploidy status. For example, all euploid embryos with negative pregnancy outcome may be placed into the negative outcome group. All aneuploid embryos may be placed into the negative outcome group. All euploid embryos with positive pregnancy outcome may be placed into the positive outcome group. In this manner, by combining the training data to indicate both the pregnancy outcome and the ploidy status, the technology described herein may seamlessly integrate to accurately predict both the ploidy status of an embryo and the viability of the embryo based on the ploidy status.

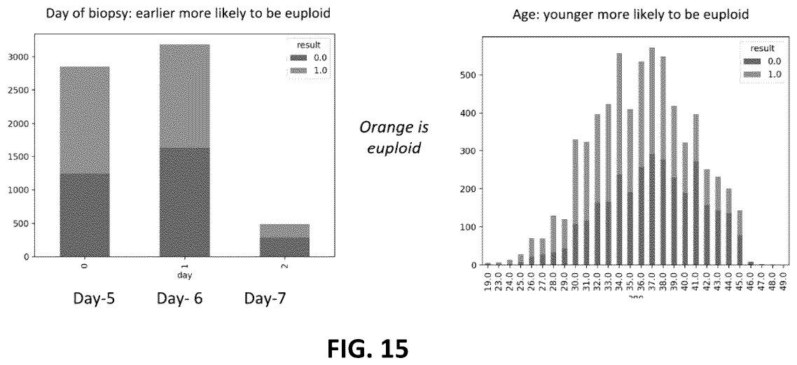

In some variations, the training dataset may include images of greater than 2000 transferred embryos with pregnancy outcomes from seven different clinics. FIG. 15 illustrates an overview of characteristics within this training dataset. For each image of an embryo with an associated ploidy status, information regarding patient age, day of transfer, and/or the like may be collected. In some variations, key parameters that may introduce a potential bias in the CNNs may be tracked. These parameters may include patient age, the day of transfer (day 5, 6 or 7). The plots in FIG. 15 show the distribution of the embryo dataset by ploidy status.

### Post-Thaw Viability Assessment

In some variations, the technology disclosed herein may additionally or alternatively be used to perform post-thaw viability assessment. For example, as described above, the technology disclosed herein may evaluate viability of embryos at the blastocyst stage. However, instead of transferring embryos at the blastocyst stage, the embryos that were considered viable may be subject to cryopreservation. Cryopreservation before transferring embryos may freeze-all cycles. This in turn may minimize the risk of hyperstimulation and may allow hormone levels to reset prior to embryo transfer. Embryos may be transferred after cryopreservation and thawing. A post-thaw viability assessment may detect embryos that may have lost at least some of their viability after freezing and thawing. That is, some embryos that may have been considered viable at the blastocyst stage may have reduced viability (e.g., have a lower level of viability or have not survived) following the process of freezing and thawing. A post-thaw viability assessment may identify such embryos.

Accordingly, the technology described herein may preform post-thaw viability analysis to identify embryos that do not survive the freeze-thaw process. However, it should be understood that such post-thaw analysis may be performed in combination with an analysis prior to freezing, or in a standalone manner. For example, in some variations, an embryo may be imaged and scored and/or ranked at multiple points in time including prior to freezing (e.g., at blastocyst stage) and after thawing. Alternatively, in some variations, an embryo may be imaged and scored and/or ranked only after thawing.

### Capturing an Image of an Embryo Post-Thaw

The plug and play software described herein such as application 106 may be used to capture one or more images of embryos post-thaw. The application may send these images to a controller such as controller 108 in FIG. 1 in real-time. The method and/or system for capturing an image of an embryo to predict post-thaw viability may be the same as the method and/or system for capturing an image of an embryo to generate a viability score (as described above) at the blastocyst stage.

### Predicting Post-Thaw Viability

The controller may implement one or more deep CNNs to predict post-thaw viability from an image. In some variations, these CNNs may be different from the CNNs described above (i.e., CNN(s) to assess embryo viability). For example, these CNNs may be trained and modeled specifically to predict post-thaw viability from images. More specifically, various architectures, hyperparameter optimization, and ensemble techniques may be implemented to train and model CNNs that may predict post-thaw viability from images. The CNNs may be trained to generate a probability score that may be indicative of whether the embryo has survived the freeze-thaw process. If the probability score is less than a threshold value, the post-thaw embryo may be deemed as not viable.

Alternatively, in addition to generating a score for an embryo, the CNNs described above may be trained to predict post-thaw viability. As described herein, a series of CNNs may be implemented to analyze and classify the images. In one example, an input image may be cropped and segmented by a CNN such as a U-Net model (such as U-Net 501 in FIG. 5). The U-Net model may be trained to segment and crop the image of the post-thaw embryo to a boundary of the post-thaw embryo. A CNN for performing quality control such as autoencoder in FIG. 6 may be trained to identify outliers using learned latent space clustering method. The autoencoder may verify whether or not an image contains a post-thaw embryo and if it does whether the post-thaw embryo has been accurately labeled. A CNN for image classification such as resnet-18 in FIG. 8 may be trained to classify the images to generate a binary output representing post-thaw viability. For example, the resnet-18 may generate output “1” indicating that a post-thaw embryo is viable and may generate output “0” indicating that a post-thaw embryo has reduced viability (e.g., lower level of viability, has not survived, etc.) following the freeze-thaw process. In some variations, CNNs trained for post-thaw viability may incorporate patient data to improve the accuracy of the prediction.

### Displaying the Output

The output may be displayed as a binary “1” and “0” and/or a “Yes” and “No.” Put differently, instead of displaying the probability and/or score of any embryo, a post-thaw viability assessment may merely indicate whether an embryo has survived the freeze-thaw process. This allows a clinician to decide whether to thaw another embryo or whether the analyzed post-thaw embryo is to be transferred.

### Training the CNNs

The data may be collected from varied sources including a consortium of clinical partners, databases comprising microscopy images of embryos, electronic medical records, and/or the like. The collected data may include microscopy images of post-thaw embryo along with electronic medical record data that may contain pregnancy outcomes for transferred post-thaw embryos, and patient data.

### Exemplary Performance Data

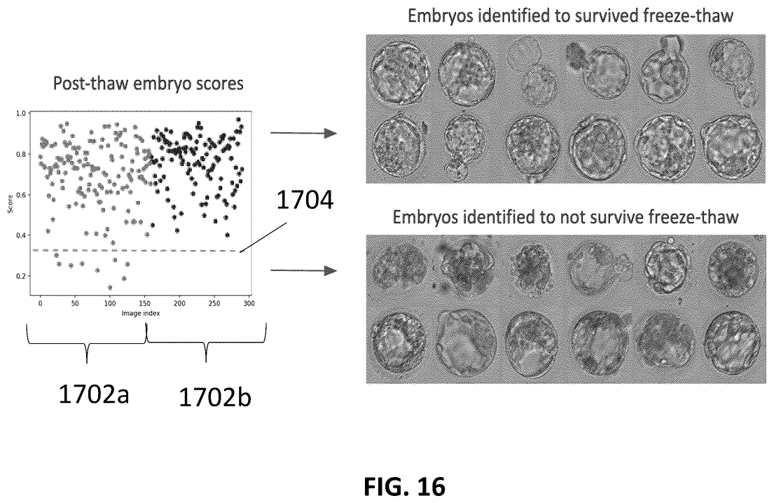

FIG. 16 illustrate post-thaw viability assessment results from a single site. The lighter colored points indicated under 1702a represent negative outcomes. The darker colored points indicated under 1702b represent positive outcomes. Line 1704 represents a threshold value. Embryos below line 1704 are embryos that do not survive the freeze-thaw process. As seen in FIG. 16, there are no false negatives. Accordingly, the present technology can use post-thaw images to identify embryos that have not survived the freeze-thaw process.

### Additional Examples of Exemplary Performance Data

A retrospective study was conducted using data collected from 11 different IVF clinics throughout the United States. Images of blastocyst stage embryos and associated metadata were collected for IVF cycles started between 2015-2020. Each clinic captured a single image of an embryo using existing hardware such as inverted microscope, stereo zoom microscope, time-lapse incubation system, etc. Images of blastocyst stage embryos were captured on day 5, 6 or 7 prior to transfer, biopsy, or cryopreservation. Approximately 5,900 blastocysts from single-embryo fresh, frozen, and frozen-euploid transfers were matched to clinical pregnancy outcomes as determined by fetal heartbeat (FHB) at 6-8 weeks. Embryos in frozen transfers were selected for warming per the standard practice at each clinic. An additional 2,600 blastocysts were matched to aneuploid (abnormal) PGT-A results.

Training data included microscopy images of embryos. Images were aggregated together and then sorted into training, validation, and test datasets. Five clinical sites provided between 600-2,000 images each with known fetal heartbeat outcomes. These images were stratified by the clinic they were obtained from, cycle type (e.g., PGT or non-PGT cycle), and outcomes. These images were also randomly split into groups for validation (e.g., 3-fold cross validation). Another five clinics provided less than 250 images each with known fetal heartbeat outcomes. All of these images were included in training. One clinic with 1000 images that were captured by a time-lapse system was reserved as a test dataset. Embryos were sorted to the positive class if they resulted in a positive fetal heartbeat, and to the negative class if they did not. To reduce the potential bias of training on only transferred embryos, non-transferred embryos that were diagnosed as aneuploid were added to the negative class.

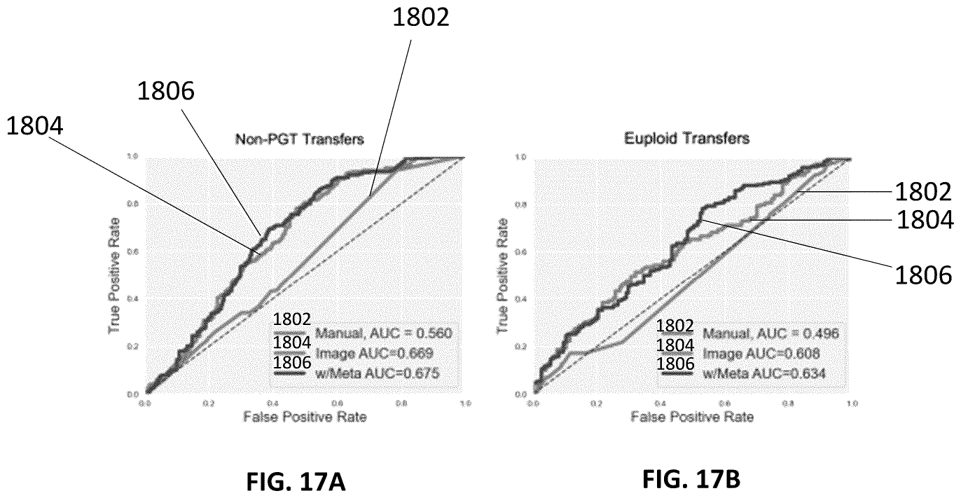

As an example, the CNNs described herein were trained using the training data described above. FIGS. 17A and 17B illustrate receiver operating characteristic (ROC) curve for example embryo transfers using CNNs described herein compared to the manual Gardner grading system. As described above, PGT-A may involve performing a biopsy to determine if the embryo has the correct number of chromosomes (euploid embryos). A non-PGT cycle may include embryo transfers without performing PGT-A. FIG. 17A illustrates ROC curves for embryo transfers without performing PGT-A. As seen in FIG. 17A, the CNNs described herein score embryos (non-PGT cycle embryos) according to their likelihood of reaching clinical pregnancy with an AUC of 0.669 using images only (trace 1804 in FIG. 17A), and 0.675 using a combination of images and patient data (trace 1806 in FIG. 17A). The AUC of the manual Gardner grading system is 0.560 (trace 1802 in FIG. 17A). Accordingly, scoring the embryos using the technology described herein shows a vast improvement in comparison to scoring the embryos using manual Gardner grading system.

FIG. 17B illustrates ROC curves for example embryos transfers after performing PGT-A. PGT-A may eliminate aneuploid embryos with abnormal number of chromosomes. Aneuploid embryos may lead to unsuccessful pregnancy. As seen in the example of FIG. 17B, the CNNs described herein score embryos (euploid embryos) according to their likelihood of reaching clinical pregnancy with an AUC of 0.608 using images only (trace 1804 in FIG. 17B), and 0.634 using a combination of images and patient data (trace 1806 in FIG. 17B). The AUC of the manual Gardner grading system is 0.496 (trace 1802 in FIG. 17B). Accordingly, similar to FIG. 17A, FIG. 17B shows that scoring the embryos using the technology described herein illustrates vast improvement in comparison to scoring the embryos using manual Gardner grading system.

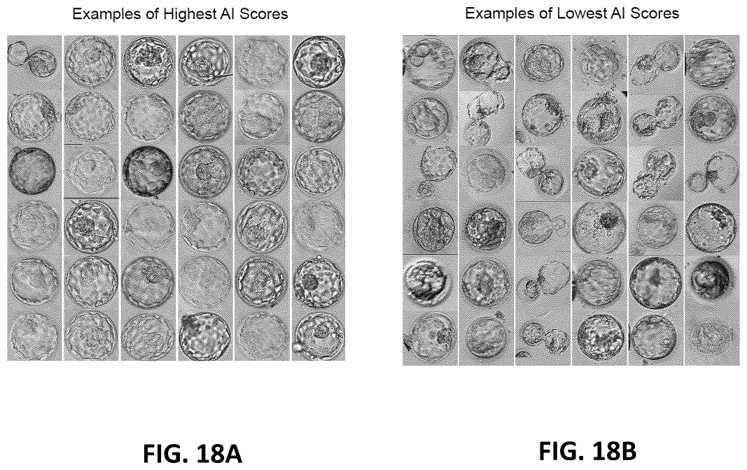

To further illustrate the performance of the technology described herein, embryos were divided into three different subgroups. The three different subgroups were top-ranked embryos with a score near 0.9, middle-ranked embryos with a score near 0.5, and lowest-ranked embryos with a score near 0.1. The embryo images for each of these subgroups were visually inspected. FIG. 18A illustrates embryo images that are top-ranked (e.g., high scores) by the technology (e.g., CNNs) described herein. As seen in FIG. 18A, the top-ranked embryos depict that top-ranked embryos are mostly fully expanded blastocysts with tightly packed and in-focus inner cellular mass, which is correctly consistent with highly viable embryos. The trophectoderm are also symmetric and in-focus with cobblestone or scalloped patterning. FIG. 18B illustrates embryo images that are lowest-ranked by the technology (e.g., CNNs) described herein. The technology described herein ranked these embryos as lowest-ranked embryos when the inner cellular mass is not visible in the image, which is correctly consistent with less viable embryos.

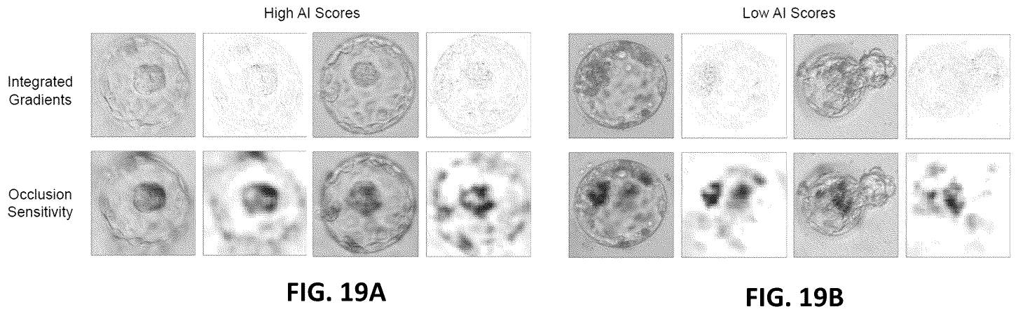

In some variations, attribution algorithms including integrated gradients and occlusion maps were used to determine whether the technology described herein was focusing on relevant features. Integrated gradients were used to determine which pixels of the image were attributed to the prediction of the technology described herein, while occlusion maps were used to show that the technology described herein is sensitive to local structure (e.g., blastocyst structure). FIG. 19A illustrates embryo images that were scored high by the technology described herein. As seen in FIG. 19A, blastocyst structures that were fully expanded with tightly packed inner cellular mass and symmetrical trophectoderm showed positive attribution and sensitivity (e.g., were classified as positive outcome images). FIG. 19B illustrates embryo images that were scored low by the technology described herein. As seen in FIG. 19B, blastocyst structures that were fragmented with cell clumping showed negative attribution and sensitivity (e.g., were classified as negative outcome images).

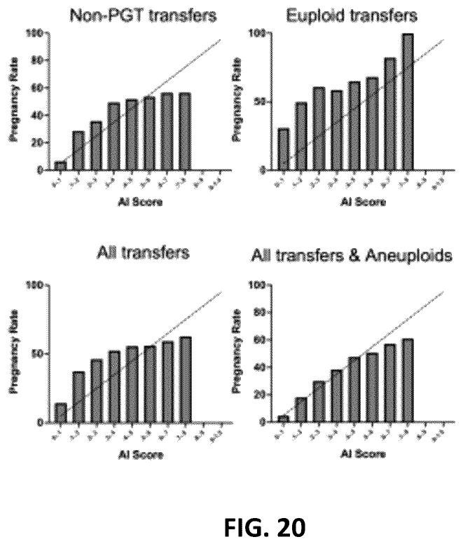

The scores assigned by the technology described herein were compared to pregnancy rates (e.g., calibration curves). FIG. 20 illustrates that the scores assigned by the technology described herein relate to the observed pregnancy rate. FIG. 20 depicts a monotonic increase in pregnancy rates for all embryos (transfers and aneuploid), all transferred embryos, and non-PGT transfers.

### Bias Removal

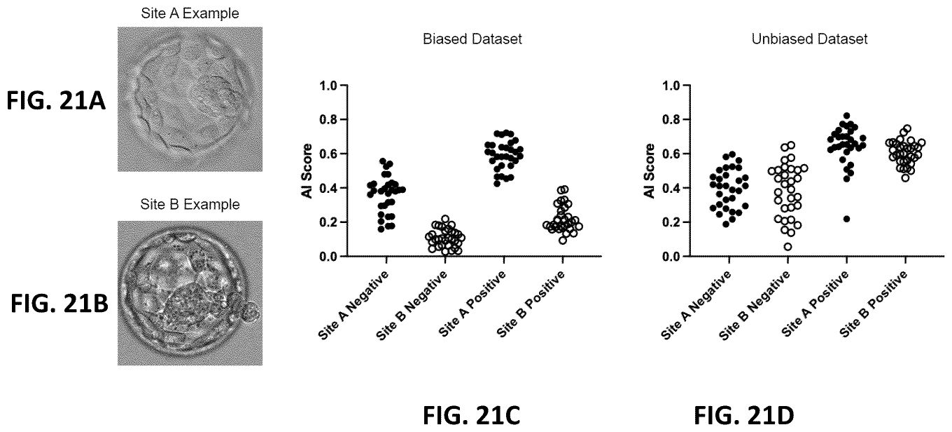

As discussed above, since images from different image capturing devices have different optics and different resolution (e.g., unique optical signature), it may be possible that a bias is introduced for images from a specific image capturing device in comparison to some other image capturing device when CNNs are trained with images from different image capturing devices. The difference in image capturing device between different clinics may lead to a biased training dataset. For example, FIGS. 21A-21D illustrate a controlled experiment to depict the biases introduced by unique optical signature of images from two different image capturing devices of two different clinics. FIG. 21A illustrates an image of an embryo captured by an image capturing device at site A (first clinic). FIG. 21B illustrates an image of an embryo captured by a different image capturing device at site B (second clinic). FIG. 21C illustrates biased dataset due to different optical signatures from site A and site B. As seen in FIG. 21C, biased datasets may result in the CNNs described herein classifying embryos from site B with lower scores as positive fetal heartbeat than embryos from site A that are classified as negative fetal heartbeat. To combat this, the CNNs may be trained by balancing a prevalence of outcome.

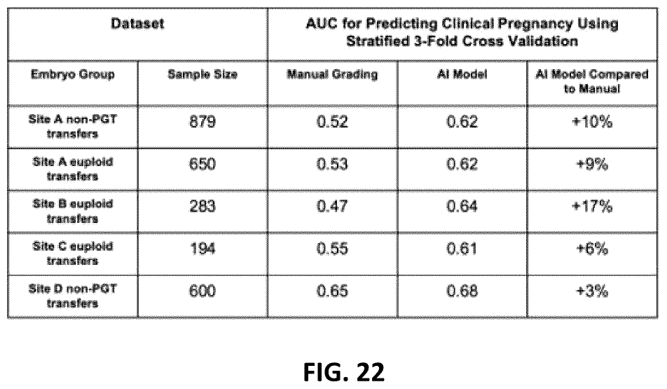

FIG. 22 illustrates a table that illustrates balancing training data based on prevalence of outcome for different clinics (e.g., site A, site B, site C, site D). The training data may be re-sampled so that every clinic has the same ratio of positive-to-negative images so as to balance the prevalence of outcome for each clinic. FIG. 21D illustrates unbiased dataset that has been balanced based on the prevalence of outcome. As seen in FIG. 21D, unbiased datasets result in the CNNs described herein classifying embryos from site B with higher scores as positive fetal heartbeat and lower scores as negative fetal heartbeat similar to classification of embryos from site A.

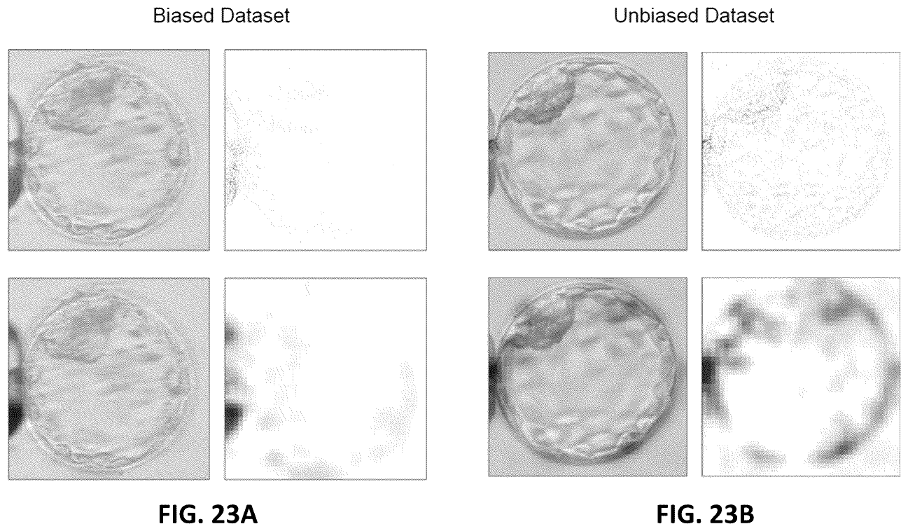

As discussed above, another source of bias may be the presence of embryo holding micropipette in an image. FIGS. 23A and 23B illustrate a controlled experiment to depict the biases introduced by the presence of micropipettes in an image. FIG. 23A illustrates that CNNs trained with a biased dataset may focus almost exclusively on the micropipette rather than the embryo. To combat this, the training data was re-sampled so that images with micropipettes have the same ratio of positive-to-negative images as images without micropipettes. FIG. 23B illustrates that training data re-sampled to balance a prevalence of outcome resulted in CNNs also focusing on the embryos in images with micropipettes. The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated. It is intended that the following claims and their equivalents define the scope of the invention.

1. A computer-implemented method for predicting viability of an embryo, the method comprising:
receiving a single image over a real-time communication link with an image capturing device;
cropping the single image to a boundary of the embryo via a first convolutional neural network;
generating a viability score for the embryo by classifying the cropped single image via at least a second convolutional neural network.

2. The method of claim 1, wherein the single image is not part of a time series of images.

3. The method of claim 1, wherein generating the viability score for the embryo is performed in response to determining that the single image depicts an embryo.

4. The method of claim 1, further comprising, in response to determining that the single image does not depict an embryo, providing an alert to a user of the image capturing device.

5. The method of claim 1, further comprising determining a probability that the embryo is a single blastocyst.

6. The method of claim 1, wherein the real-time communication link is provided by an application executed on a computing device communicably coupled to the image capturing device.

7. The method of claim 6, wherein the application causes a display on the computing device to display a capture button.

8. The method of claim 7, wherein in response to a user selecting the capture button, the image capturing device captures the first single image of the embryo.

9. The method of claim 1, wherein the viability score represents a likelihood of the embryo reaching clinical pregnancy.

10. The method of claim 1, wherein the viability score represents a likelihood of the embryo reaching live birth.

11. The method of claim 1, wherein the likelihood of the embryo reaching clinical pregnancy is associated with an outcome of a fetal cardiac activity.

12. The method of claim 1, wherein the viability score is based at least in part on data associated with a patient.

13. The method of claim 12, wherein the data includes at least one of age, body mass index, day of image capture, and donor status.

14. The method of claim 1, further comprising storing the viability score in a database.

15. The method of claim 1, further comprising communicating the viability score to at least one of a patient and a clinician.

16. The method of claim 1, further comprising predicting, via a fourth convolutional neural network, whether the embryo is euploid or aneuploid.

17. The method of claim 16, wherein predicting whether the embryo is euploid or aneuploid depends at least in part on data associated with a subject.

18. The method of claim 17, wherein the data is at least one of age and day of biopsy.

19. The method of claim 17, further comprising:
generating a ploidy outcome based on whether the embryo is euploid or aneuploid; and
updating at least the fourth convolutional neural network based at least in part on the ploidy outcome and the data.

20. The method of claim 1, wherein the embryo is to undergo at least one of biopsy and freezing, and wherein the method further comprises receiving the single image of the embryo prior to biopsy or freezing, and determining viability of the embryo prior to at least one of biopsy and freezing.

21. The method of claim 1, wherein the embryo has been frozen and thawed, and wherein the method further comprises receiving the single image of the embryo post-thaw, and determining viability of the embryo post-thaw via the second convolutional neural network.

22. The method of claim 21, wherein determining viability of the embryo post-thaw comprises classifying the single image into either a first class indicating that the embryo has survived post-thaw, or a second class indicating that the embryo has not survived post-thaw.

23. The method of claim 1, further comprising receiving a plurality of single images, each single image depicting a respective embryo of a plurality of embryos, generating a viability score for each embryo by classifying each single image via the second convolutional neural network, and ranking the plurality of embryos based on the viability scores for the plurality of embryos.

24-48. (canceled)
